(19) United States
(12) Patent Application Publication
(10) Pub. No.: US 2023/0285764 A1
(43) Pub. Date: Sep. 14, 2023

(54) WEARABLE MEDICAL SYSTEM (WMS) IMPLEMENTING WEARABLE CARDIOVERTER DEFIBRILLATOR (WCD) AND RECORDING ECG OF PATIENT IN REGULAR MODE AND IN RICH MODE

(71) Applicant: West Affum Holdings DAC, Dublin (IE)

(72) Inventors: Ronald K. Rowbotham, Lake Forest Park, WA (US); Dallas E. Meeker, Kirkland, WA (US); Gregory T. Kavounas, Bellevue, WA (US)

(73) Assignee: West Affum Holdings DAC, Dublin (IE)

(21) Appl. No.: 18/178,362

(22) Filed: Mar. 3, 2023

Related U.S. Application Data
(60) Provisional application No. 63/318,285, filed on Mar. 9, 2022.

(51) Int. Cl. A61N 1/39; A61B 5/00; A61B 5/33; A61B 5/335; A61B 5/364

(52) U.S. Cl. CPC A61N 1/3904; A61B 5/0006; A61B 5/33; A61B 5/335; A61B 5/364; A61N 1/3975

(57) ABSTRACT

In embodiments, a wearable medical system (“WMS”) implements a wearable cardioverter defibrillator (“WCD”) that senses and samples a patient's ECG signals. In a regular mode, the WMS produces a first set of ECG values, which can be the minimum needed for a WCD operation. In a second or rich mode of operation, the WMS produces a second set of ECG values, more numerous than the first set. The rich mode can be implemented by sampling the ECG signal faster, and/or not ignoring ECG signals in channels that are ignored in the regular mode, and/or by having more ECG sensing electrodes than the minimum needed for the WCD operation. The WMS stores the first set and the second set, and uses either one to determine whether defibrillation is needed. In addition, it communicates them to another device. In embodiments, support structures for a WMS have multiple ECG electrodes for just sensing.

### CROSS-REFERENCE TO RELATED APPLICATIONS

This patent application claims priority from U.S. provisional patent application Ser. No. 63/318,285, filed on Mar. 9, 2022, which is hereby incorporated by reference for all purposes.

### BACKGROUND

A wearable medical system (“WMS”) is an advanced form of a medical system. A WMS typically includes one or more wearable components that a patient can wear or carry, and possibly other components that can be portable, or stationary such as base station and/or an electric charger. The WMS may also include one or more associated software packages, such as software applications (“apps”), which can be hosted by the wearable component, and/or by a mobile device, and/or by a remote computer system that is accessible via a communications network such as the internet, and so on.

A WMS typically includes a sensor that can sense when a parameter of the patient is problematic, and cause the WMS to initiate an appropriate action. The appropriate action could be for the WMS to communicate with the patient or even with a bystander, to transmit an alert to a remotely located clinician, and to even administer treatment or therapy to the patient by itself. A WMS may actually include more than one sensor, which may sense more than one parameter of the patient. The multiple parameters may be used for determining whether or not to administer the treatment or therapy, or be suitable for detecting different problems and/or for administering respectively different treatments or therapies to the patient.

A WMS may also include the appropriate components for implementing a wearable cardioverter defibrillator (“WCD”), a pacer, and so on. Such a WMS can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time, such as 10 minutes. Some observers may have thought that SCA is the same as a heart attack, but it is not. For such patients, an external cardiac defibrillator can deliver a shock through the heart, and restore its normal rhythm. The problem is that it is hard for an external cardiac defibrillator to be brought to the patient within that short time. One solution, therefore, is for such patients to be given a WMS that implements a WCD. This solution is at least temporary and, after a while such as two months, the patient may instead receive a surgically implantable cardioverter defibrillator (“ICD”), which would then become a permanent solution.

A WMS that implements a WCD typically includes a harness, vest, belt, or other garment that the patient is to wear. The WMS system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMS, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving their life.

All subject matter discussed in this Background section of this document is not necessarily prior art, and may not be presumed to be prior art simply because it is presented in this Background section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventors. This approach in and of itself may also be inventive.

### SUMMARY

In embodiments, a wearable medical system (“WMS”) for an ambulatory patient implements a wearable cardioverter defibrillator (“WCD”) that senses the patient's ECG signals.

In a first or regular mode of operation, the WMS samples the sensed ECG signals to produce a first set of ECG values, the first set having a first number of ECG values per unit time. The first set can be the minimum needed for a WCD operation.

In a second or rich mode of operation, the WMS samples the sensed ECG signals to produce a second set of ECG values, the second set having a second number of ECG values per unit time. The second number is larger than the first number, sometimes much larger. The rich mode can be implemented by sampling the ECG signal faster, and/or not ignoring ECG signals in channels that are ignored in the regular mode, and/or by having additional ECG sensing electrodes than the minimum needed for the WCD operation.

The WMS stores the first set and the second set, and can use either one to determine whether defibrillation is needed. In addition, it communicates them to another device.

In embodiments, support structures for a WMS have multiple ECG electrodes for just sensing the ECG.

An advantage and/or benefit can be that, when the rich mode is implemented with additional ECG sensing electrodes, additional vectors are created that provide channels, and therefore there is a better chance for finding a non-noisy ECG channel in the regular operation.

An additional advantage and/or benefit can be that, when operating in the rich mode, the WCD can better distinguish ventricular tachycardias that are shockable from atrial tachycardias that are not shockable. As such the WMS might not administer a shock that is not needed, and which in fact could be harmful to the patient.

Another advantage and/or benefit can be that the data collected from the rich ECG mode can help with the further study of the deterioration process of a heart transitioning from normal sinus rhythm to fibrillation. A further advantage may result in learning from such data and applying it enough to recognize where such deterioration starts, and communicate to the patient while they are still conscious, contact a remote health care attendant, and so on.

One more advantage and/or benefit can be that benefits of a 12-lead ECG can be had by patients of a WCD. For instance, a WMS according to embodiments may be able to further diagnose a) poor blood flow to the heart muscle (ischemia), b) heart attack, and c) abnormalities of the heart such as heart chamber enlargement and abnormal conduction. Moreover, a 12-lead ECG can be had immediately after defibrillation, giving a picture of the heart as it hopefully restarts.

As such, it will be appreciated that embodiments have utility, and in fact may cause results that are larger than the sum of their individual parts.

These and other features and advantages of the claimed invention will become more readily apparent in view of the embodiments described and illustrated in this specification, namely in this written specification and the associated drawings.

### BRIEF DESCRIPTION OF THE DRAWINGS

FIG. 1 is a diagram of sample components of a wearable medical system (“WMS”) that implements a wearable cardioverter defibrillator (“WCD”), and which is made according to embodiments.

FIG. 2A is a diagram showing a view of the inside of a sample garment embodiment that can be a support structure of a WMS that implements a WCD, such as that of FIG. 1.

FIG. 2B is a diagram showing a view of the outside of the sample garment of FIG. 2A.

FIG. 2C is a diagram showing a front view of how the sample garment of FIGS. 2A and 2B is intended to be worn by a patient.

FIG. 2D is a diagram showing a back view of how the sample garment of FIGS. 2A and 2B is intended to be worn by a patient.

FIG. 3 is a diagram showing a partial front view of another patient wearing a sample garment embodiment of an alternate style as worn by a patient, and which can be a support structure of a WMS that implements a WCD such as that of FIG. 1.

FIG. 4 is a diagram showing sample embodiments of electronic components of a WMS that implements a WCD, and which can be used with the garment of FIG. 2A or of FIG. 3.

FIG. 5 is a diagram showing sample components of a unit of FIG. 1, which is made according to embodiments.

FIG. 6 is a conceptual diagram showing an embodiment where ECG values recorded during a rich mode of operation include all of the ECG values recorded during a regular mode of operation.

FIG. 7 is a conceptual diagram showing an embodiment where ECG values recorded during a rich mode of operation include some but not all of the ECG values recorded during a regular mode of operation.

FIG. 8 is a conceptual diagram showing an embodiment where ECG values recorded during a rich mode of operation include none of the ECG values during a regular mode of operation.

FIG. 9 shows and compares two time diagrams with sampled and recorded ECG values in a regular mode of operation and in a rich mode of operation, and further depicts sectors of the memory where these ECG values may be stored according to embodiments.

FIG. 10 is a time diagram showing embodiments where ECG values in the rich mode of FIG. 9 is sometimes recorded instead of ECG values in the regular mode of FIG. 9.

FIG. 11A is a conceptual diagram showing that a rich ECG mode of recording ECG values can be switched on, plus sample events that might cause this to happen, according to embodiments.

FIG. 11B is a conceptual diagram showing that a rich ECG mode of recording ECG values can be switched off, plus sample events that might cause this to happen, according to embodiments.

FIG. 12 shows related time diagrams to illustrate that more ECG values are sampled and recorded in a rich mode with a faster rich sampling rate, than in a regular mode with a regular sampling rate.

FIG. 13 is a diagram that illustrates conceptually how multiple ECG sensing electrodes may be used for sensing ECG signals along different channels in a WMS that implements a WCD according to embodiments, to collect ECG values in a regular mode and/or in a rich mode.

FIG. 14 is a diagram showing a support structure that supports multiple ECG sensing electrodes in addition to defibrillation electrodes, according to embodiments.

FIG. 15 shows a first table of locations of ECG sensing electrodes on a support structure of a WMS that implements a WCD according to embodiments, and a second table of potential vectors that define channels along which ECG signals may be sensed to generate a rich set of ECG values, according to embodiments.

FIG. 16 is a diagram of a sample support structure for a WMS that is implemented using belts according to embodiments.

FIG. 17A is an anterior (front) view of a sample support structure for a WMS that is implemented by a vest, according to embodiments.

FIG. 17B is a posterior (rear) view of the support structure of FIG. 17A.

FIG. 17C is a superior (perspective) view of the support structure of FIG. 17A.

FIG. 18 is a flowchart for illustrating sample methods according to embodiments.

FIG. 19 is a diagram showing a support structure that supports multiple ECG sensing electrodes, according to embodiments.

FIG. 20 is a diagram of a sample support structure for a WMS that is implemented using belts according to embodiments.

FIG. 21A is an anterior (front) view of a sample support structure for a WMS that is implemented by a vest, according to embodiments.

FIG. 21B is a posterior (rear) view of the support structure of FIG. 21A.

FIG. 21C is a superior (perspective) view of the support structure of FIG. 21A.

### DETAILED DESCRIPTION

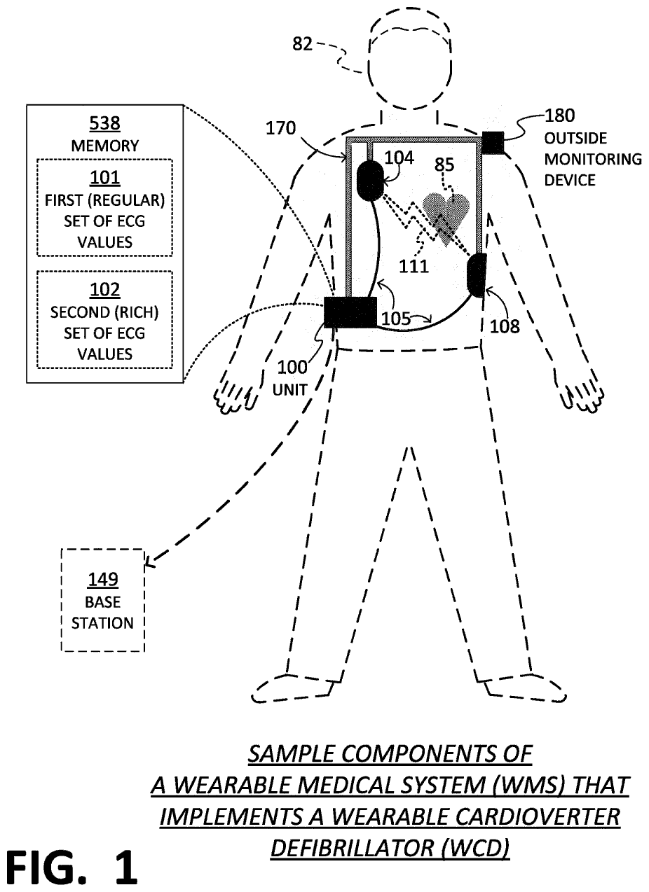

Referring to FIG. 1, in embodiments, a wearable medical system (“WMS”) for an ambulatory patient implements a wearable cardioverter defibrillator (“WCD”) that senses the patient's ECG signals and can sample the sensed ECG signals in a regular mode and/or in a rich mode.

A wearable medical system (“WMS”) that implements a wearable cardioverter defibrillator (“WCD”) according to embodiments may protect a patient by electrically restarting their heart if needed. Such a WMS may have a number of components. These components can be provided separately as modules that can be interconnected, or can be combined with other components, and so on. Examples are now described.

FIG. 1 depicts a patient 82. The patient 82 may also be referred to as the person 82 and/or wearer 82, since the patient 82 is wearing components of the WMS. The patient 82 is ambulatory, which means that, while wearing the wearable component(s) of the WMS, the patient 82 can (while physically able) walk around, be in a vehicle, and so on. In other words, the patient 82 is not necessarily bed-ridden. It should also be noted that referring to the patient as “ambulatory” in this document does not imply that the patient must be walking while wearing the WMS. Rather, the term “ambulatory” merely indicates that the patient may walk around while wearing the WMS. Indeed, an ambulatory patient who experiences an SCA is highly unlikely to be walking while experiencing the SCA even though the patient may have been walking just prior to the SCA. While the patient 82 may be considered to be also a “user” of the WMS, this definition is not exclusive to the patient 82. For instance, a user of the WMS may also be a clinician such as a doctor, nurse, emergency medical technician (EMT), or other similarly tasked and/or empowered individual or group of individuals. In some cases, a user may even be a bystander. The particular context of these and other related terms within this description should be interpreted accordingly.

A WMS that implements a WCD according to embodiments can be configured to defibrillate the patient who is wearing the designated components of the WMS. Defibrillating can be by the WMS delivering an electrical charge to the patient's body in the form of an electric shock. The electric shock can be delivered in one or more pulses.

In particular, FIG. 1 also depicts components of a WMS that implements a WCD and is made according to embodiments. One such component is a support structure 170 that is wearable by the ambulatory patient 82. Accordingly, the support structure 170 can be configured to be worn by the ambulatory patient 82 for at least several hours per day, and also during the night. That, for at least several days, and maybe even a few months. It will be understood that the support structure 170 is shown only generically in FIG. 1, and in fact partly conceptually. FIG. 1 is provided merely to illustrate concepts about the support structure 170, and is not to be construed as limiting how the support structure 170 is implemented, or how it is worn.

The support structure 170 can be implemented in many different ways. For example, it can be implemented in a single component or a combination of multiple components. In embodiments, the support structure 170 could include a vest, a half-vest, a garment, etc. In such embodiments such items can be worn similarly to analogous articles of clothing. In embodiments, the support structure 170 could include a harness, one or more belts or straps, etc. In such embodiments, such items can be worn by the patient around the torso, hips, over the shoulder, etc. In embodiments, the support structure 170 can include a container or housing, which can even be waterproof. In such embodiments, the support structure can be worn by being attached to the patient's body by adhesive material, for example as shown and described in U.S. Pat. No. 8,024,037. The support structure 170 can even be implemented as described for the support structure of US Pat. App. No. US2017/0056682, which is incorporated herein by reference. Of course, in such embodiments, the person skilled in the art will recognize that additional components of the WMS can be in the housing of a support structure instead of being attached externally to the support structure, for example as described in the US2017/0056682 document. There can be other examples.

The embodiments of FIG. 1 include a sample unit 100. In embodiments, the unit 100 is configured to be maintained on a body of the ambulatory patient 82, when the support structure 170 is worn by the ambulatory patient. This can be accomplished in number of ways, for instance the unit 100 can be attachable to the support structure 170 itself. In embodiments, the unit 100 is sometimes called a main electronics module. In embodiments, the unit 100 implements an external defibrillator. In embodiments, the unit 100 implements an external pacer instead of, or in addition to, an external defibrillator. In embodiments that include a pacer, the WMS may detect when the patient's heart rhythm slows down or when the patient has asystole, and the pacer may pace to increase the heart rate. In such embodiments, the WMS may pace the patient first, and hopefully not have to resort to the full intervention of defibrillation. Of course, if the patient does not respond to the pacing and their heart rhythm deteriorates further, the WMS may then later cause one or more defibrillation shocks to be delivered.

The embodiments of FIG. 1 also include sample therapy electrodes 104, 108, which are electrically coupled to unit 100 via electrode leads 105. The therapy electrodes 104, 108 are also called defibrillation electrodes or just electrodes. The therapy electrodes 104, 108 can be configured to be worn by the patient 82 in a number of ways. For instance, the unit 100 and the therapy electrodes 104, 108 can be coupled to the support structure 170, directly or indirectly. In other words, the support structure 170 can be configured to be worn by the ambulatory patient 82 so as to maintain at least one of the therapy electrodes 104, 108 on the body of the ambulatory patient 82, while the patient 82 is moving around, etc. The therapy electrodes 104, 108 can be thus maintained on the body by being attached to the skin of the patient 82, simply pressed against the skin directly or through garments, etc. In some embodiments the therapy electrodes 104, 108 are not necessarily pressed against the skin, but become biased that way upon sensing a condition that could merit intervention by the WMS. In addition, many of the components of the unit 100 can be considered coupled to the support structure 170 directly, or indirectly via at least one of the therapy electrodes 104, 108.

When the therapy electrodes 104, 108 make good electrical contact with the body of the patient 82, the unit 100 can administer, via the therapy electrodes 104, 108, a brief, strong electric pulse 111 through the body. The pulse 111 is also known as defibrillation pulse, shock, defibrillation shock, therapy, electrotherapy, therapy shock, etc. The pulse 111 is intended to go through and restart the heart 85, in an effort to save the life of the patient 82. The defibrillation pulse 111 can have an energy suitable for its purpose, such as at least 100 Joule (“J”), 200 J, 300 J, and so on. For pacer embodiments, the pulse 111 could alternately be depicting a pacing pulse. At least some of the stored electrical charge can be caused to be discharged via at least two of the therapy electrodes 104, 108 through the ambulatory patient 82, so as to deliver to the ambulatory patient 82 a pacing sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 10 J, 5 J, usually about 2 J, and so on. The pacer therefore is delivering current to the heart to start a heartbeat. In either case, the pulse 111 has a waveform suitable for this purpose.

A prior art defibrillator typically decides whether to defibrillate or not based on an ECG signal of the patient. However, the unit 100 may initiate defibrillation, or hold-off defibrillation, based on a variety of inputs, with the ECG signal merely being one of these inputs.

A WMS that implements a WCD according to embodiments can collect data about one or more parameters of the patient 82. For collecting such data, the WMS may optionally include at least an outside monitoring device 180. The device 180 is called an “outside” device because it could be provided as a standalone device, for example not within the housing of the unit 100. The device 180 can be configured to sense or monitor at least one local parameter. A local parameter can be a parameter of the patient 82, or a parameter of the WMS, or a parameter of the environment, as described later in this document.

For some of these parameters, the device 180 may include one or more sensors or transducers. Each one of such sensors can be configured to sense a parameter of the patient 82, or of the environment, and to render an input responsive to the sensed parameter. In some embodiments the input is quantitative, such as values of a sensed parameter; in other embodiments the input is qualitative, such as informing whether or not a threshold is crossed, and so on. Such inputs about the patient 82 are also called physiological inputs and patient inputs. In embodiments, a sensor can be construed more broadly, as encompassing more than one individual sensors.

Optionally, the device 180 is physically coupled to the support structure 170. In addition, the device 180 may be communicatively coupled with other components that are coupled to the support structure 170, such as with the unit 100. Such communication can be implemented by the device 180 itself having a communication module, as will be deemed applicable by a person skilled in the art in view of this description.

A WMS that implements a WCD according to embodiments preferably includes sensing electrodes, which can sense an ECG of the patient. In embodiments, the device 180 stands for such sensing electrodes. In those embodiments, the sensed parameter of the patient 82 is the ECG of the patient, the rendered input can be time values of a waveform of the ECG signal, and so on.

In embodiments, one or more of the components of the shown WMS may be customized for the patient 82. This customization may include a number of aspects. For instance, the support structure 170 can be fitted to the body of the patient 82. For another instance, baseline physiological parameters of the patient 82 can be measured for various scenarios, such as when the patient is lying down (various orientations), sitting, standing, walking, running, and so on. These baseline physiological parameters can be the heart rate of the patient 82, motion detector outputs, one for each scenario, etc. The measured values of such baseline physiological parameters can be used to customize the WMS, in order to make its diagnoses more accurate, since patients' bodies differ from one another. Of course, such parameter values can be stored in a memory of the WMS, and so on. Moreover, a programming interface can be made according to embodiments, which receives such measured values of baseline physiological parameters. Such a programming interface may input automatically these in the WMS, along with other data.

The support structure 170 is configured to be worn by the ambulatory patient 82 so as to maintain the therapy electrodes 104, 108 on a body of the patient 82. As mentioned before, the support structure 170 can be advantageously implemented by clothing or one or more garments. Such clothing or garments do not have the function of covering a person's body as a regular clothing or garments do, but the terms “clothing” and “garment” are used in this art for certain components of the WMS intended to be worn on the human body in the same way as clothing and garments are. In fact, such clothing and garments of a WMS can be of different sizes for different patients, and even be custom-fitted around the human body. And, regular clothing can often be worn over portions or all of the support structure 170. Examples of the support structure 170 are now described.

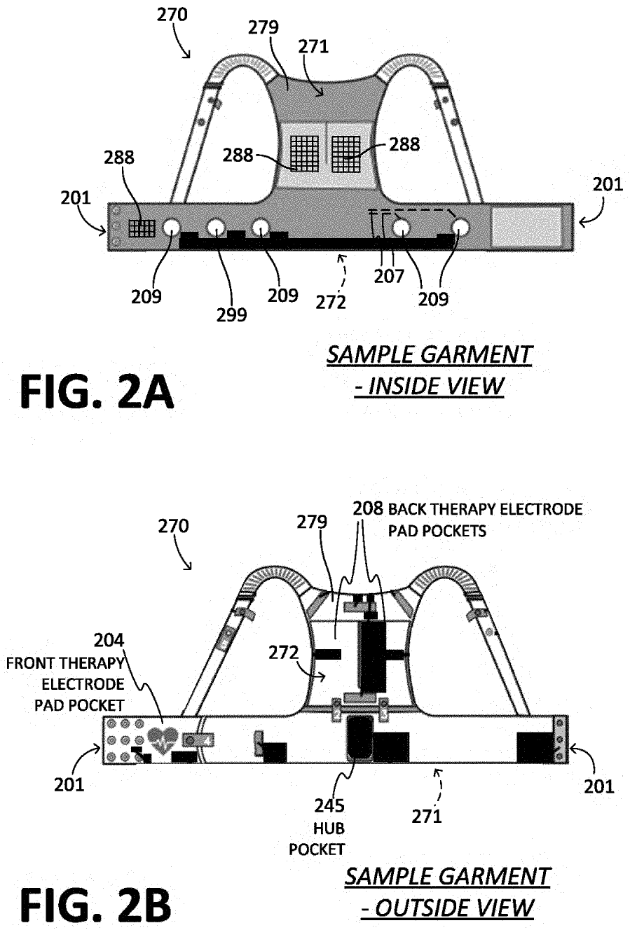

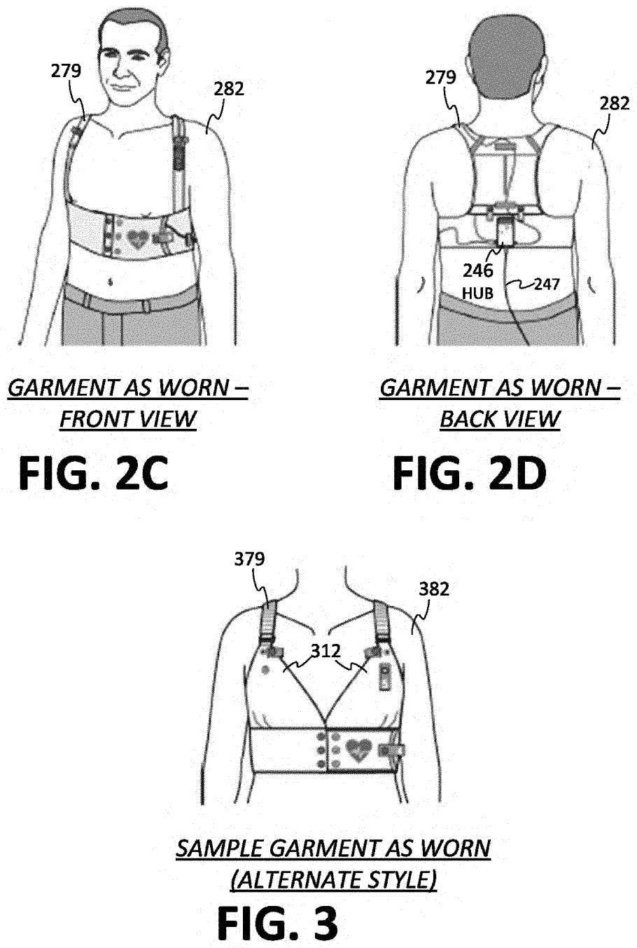

FIG. 2A shows a support structure 270 of a WMS that implements a WCD, such as the support structure 170 of FIG. 1. The support structure 270 is implemented by a vest-like wearable garment 279 that is shown flat, as if placed on a table. The inside side 271 of the garment 279 is seen as one looks at the diagram from the top, and it is the side contacting the body of the wearer when the garment 279 is worn. The outside side 272 of the garment 279 is opposite the inside side 271. To be worn, tips 201 can be brought together while surrounding the torso, and affixed to each other, either at their edges or partly overlapping. Appropriate mechanisms can hold together the tips 201, such as hooks and loops, Velcro® material, and so on.

The garment 279 can be made of suitable combinations of materials, such as fabric, linen, plastic, and so on. In places, the garment 279 can have two adjacent surfaces for defining between them pockets for the pads of the electrodes, for enclosing the leads or wires of the electrodes, and so on. Moreover, in FIG. 2A one can see meshes 288 which are the interior side of pockets accessible from the outside. The meshes can be made from flexible material such as loose netting, and so on.

ECG signals in a WMS that implements a WCD may sometimes include too much electrical noise for analyzing the ECG signal. To ameliorate the problem, multiple ECG sensing electrodes are provided in embodiments. These multiple ECG sensing electrodes, taken pairwise, define different vectors that define channels for sensing ECG signals along different ECG channels. These different ECG channels therefore present alternative options for analyzing the patient's ECG signal. The patient impedance along each ECG channel may also be sensed, and thus be part of the patient input.

In the example of FIG. 2A, multiple ECG sensing electrodes 209 are provided, which can be seen protruding from the inside surface of the garment 279. These ECG sensing electrodes 209 can be affixed to the inside surface of the garment 279, while their leads or wires 207 can be located mostly or completely within the garment 279. These ECG sensing electrodes 209 are intended to contact the skin of the person when the garment 279 is worn, and can be made from suitable material for good electrical contact. Such a material can be a metal, such as silver. An additional ECG-sensing electrode 299 may play the role of a Right Leg Drive (“RLD”) in the ECG analysis. In this context “RLD” is a custom electrical term, and embodiments do not require that the electrode 299 be actually placed on a leg of the patient.

FIG. 2B shows the outside side 272 of the garment 279. One can appreciate that pockets are included that are accessible from the outside, such as a hub pocket 245. In addition a pocket 204 is provided for a front therapy electrode pad, plus two pockets 208 are provided for two back therapy electrode pads. The pads of the therapy electrodes can be placed in the pockets 204, 208, and contact the skin of the patient through the respective meshes 288 that were seen in FIG. 2A. The electrical contact can be facilitated by conductive fluid that can be deployed in the area, when the time comes for a shock.

FIG. 2C is a diagram showing a front view of how the garment 279 would be worn by a patient 282. It will be appreciated that the previously described ECG sensing electrodes 209, 299 of FIG. 2A are maintained against the body of the patient 282 from the inside side of the garment 279, and thus are not visible in FIG. 2C.

FIG. 2D is a diagram showing the back view of FIG. 2C. A hub 246 has been placed in the hub pocket 245 that is shown in FIG. 2B. A cable 247 emerges from the hub 246, which can be coupled with a unit for the system, as described later in this document.

FIGS. 2A-2D do not show any physical support for a unit such as the unit 100 of FIG. 1. In these embodiments, such a unit may be carried in a purse, on a belt, by a strap over the shoulder, or additionally by further adapting the garment 279, and so on.

FIG. 3 is a diagram showing a partial front view of another patient 382 wearing another garment 379. The garment 379 is of an alternate style than the garment 279, in that it further includes breast support receptacles 312, as was described for instance in U.S. Pat. No. 10,926,080. This style of garment may be more comfortable if the patient 382 is a woman.

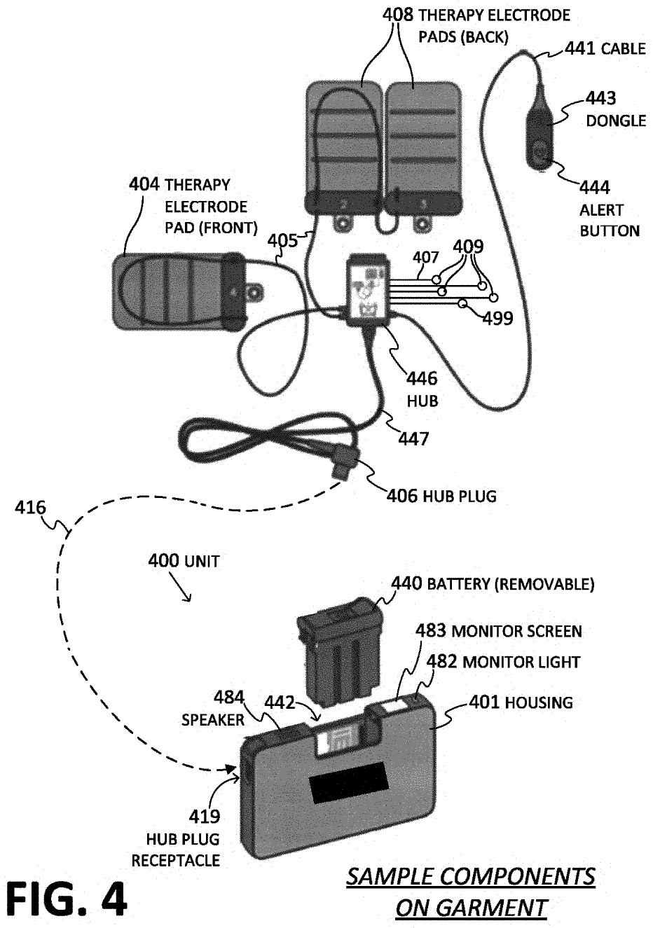

FIG. 4 shows sample electronic components that can be used with the garments 279, 379. The components of FIG. 4 include a unit 400, shown at the lower portion of FIG. 4. The unit 400 includes a housing 401, and a hub plug receptacle 419 at the housing 401.

The unit 400 includes a battery opening 442 at the housing 401. The battery opening 442 is configured to receive a removable battery 440. A system according to embodiments can have two identical such batteries 440, one plugged into the housing 401 while another one (not shown) is being charged by a charger (not shown). The batteries can then be interchanged when needed.

The unit 400 also includes devices for implementing a user interface. In this example, these devices include a monitor light 482, a monitor screen 483 and a speaker 484. Additional devices may include a vibrating mechanism, and so on.

The unit 400 can implement many of the functions of the unit 100 of FIG. 1. In the embodiment of FIG. 4, however, some of the functions of the unit 100 are implemented instead by a separate hub 446, which can be connected to the unit 400. The hub 446 is smaller and lighter than the unit 400, and can accommodate multiple electrical connections to other components of FIG. 4. A cable 447, similar to the cable 247 of FIG. 2D, emerges from the hub 446 and terminates in a hub plug 406. The hub plug 406 can be plugged into the hub plug receptacle 419 of the unit 400 according to an arrow 416.

ECG sensing electrodes 409, 499, plus their wires or leads 407 are further shown conceptually in FIG. 4 for completeness. The wires or leads 407 that can be configured to be coupled to the hub 446.

The components of FIG. 4 also include the therapy electrode pads 404, 408. The therapy electrode pad 404 can be inserted into the pocket 204 of FIG. 2B, while the therapy electrode pads 408 can be inserted into the pockets 208 of FIG. 2B. The shock is generated between the therapy electrode pad 404 and the therapy electrode pads 408 taken together. Indeed, the therapy electrode pads 408 are electrically connected to each other. The therapy electrode pads 404, 408, have leads 405, which can be configured to be coupled to the hub 446.

The components of FIG. 4 further include a dongle 443 with an alert button 444. The dongle 443 can be configured to be coupled to the hub 446 via a cable 441. The alert button 444 can be used by the patient to give emergency input to the WMS. For instance, the alert button 444 can be used by the patient to notify the system that the patient is actually alive and an imminent shock is not actually needed, which may otherwise happen in the event of a false positive detection of a shockable heart rhythm of the patient.

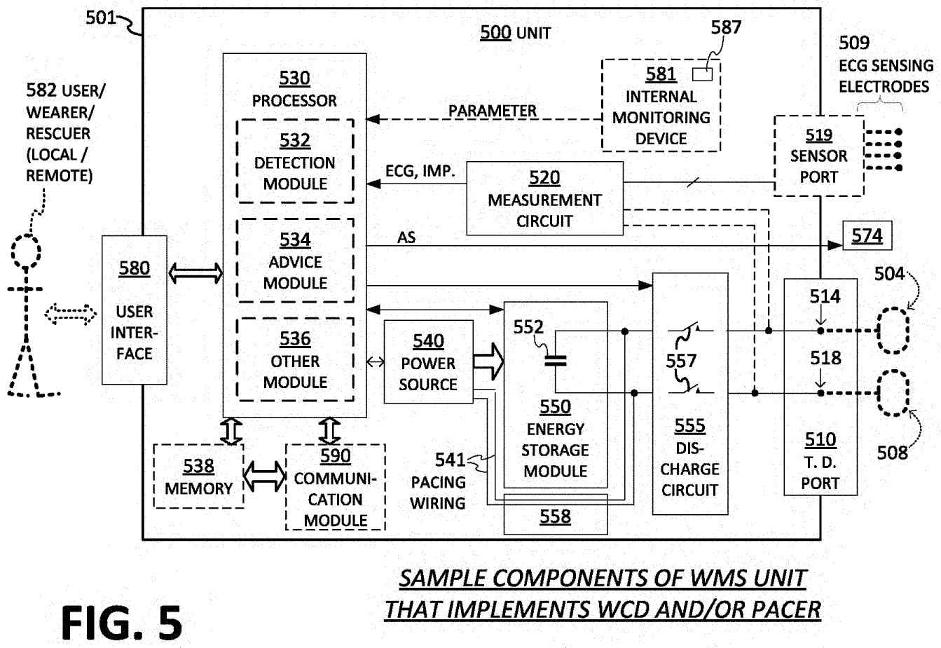

FIG. 5 shows a sample unit 500, which could be the unit 100 of FIG. 1. The unit 500 implements an external defibrillator and/or a pacer. The sample unit 500 thus combines the functions of the unit 400 and of the hub 446 of FIG. 4. The components shown in FIG. 5 can be provided in a housing 501, which may also be referred to as casing 501.

The unit 500 may include a user interface (UI) 580 for a user 582. User 582 can be the patient 82, also known as patient 582, also known as the wearer 582. Or, the user 582 can be a local rescuer at the scene, such as a bystander who might offer assistance, or a trained person. Or, the user 582 might be a remotely located trained caregiver in communication with the WMS, such as a clinician.

The user interface 580 can be made in a number of ways. The user interface 580 may include output devices, which can be visual, audible or tactile, for communicating to a user by outputting images, sounds or vibrations. Images, sounds, vibrations, and anything that can be perceived by user 582 can also be called human-perceptible indications. As such, an output device according to embodiments can be configured to output a human-perceptible indication (HPI). Such HPIs can be used to alert the patient, sound alarms that may be intended also for bystanders, and so on. There are many instances of output devices. For example, an output device can be a light that can be turned on and off, a screen to display what is sensed, detected and/or measured, and provide visual feedback to the local rescuer 582 for their resuscitation attempts, and so on. Another output device can be a speaker, which can be configured to issue voice prompts, alerts, beeps, loud alarm sounds and/or words, and so on. These can also be for bystanders, when defibrillating or just pacing, and so on. Examples of output devices were the monitor light 482, the monitor screen 483 and the speaker 484 of the unit 400 seen in FIG. 4.

The user interface 580 may further include input devices for receiving inputs from users. Such users can be the patient 82, 582, perhaps a local trained caregiver or a bystander, and so on. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock, or of pacing pulses. In particular, in some embodiments a speaker of the WMS is configured to output a warning prompt prior to an impending or planned defibrillation shock or a pacing sequence of pacing pulses being caused to be delivered, and the cancel switch is configured to be actuated by the ambulatory patient 82 in response to the warning prompt being output. In such embodiments, the impending or planned defibrillation shock or pacing sequence of the pacing pulses is not caused to be delivered. An example of a cancel switch was the alert button 444 seen in FIG. 4.

The unit 500 may include an internal monitoring device 581. The device 581 is called an “internal” device because it is incorporated within the housing 501. The monitoring device 581 can sense or monitor patient parameters such as patient physiological parameters, system parameters and/or environmental parameters, all of which can be called patient data. In other words, the internal monitoring device 581 can be complementary of, or an alternative to, the outside monitoring device 180 of FIG. 1. Allocating which of the parameters are to be monitored by which of the monitoring devices 180, 581 can be done according to design considerations. The device 581 may include one or more sensors, as also described elsewhere in this document.

Patient parameters may include patient physiological parameters. Patient physiological parameters may include, for example and without limitation, those physiological parameters that can be of any help in detecting by the WMS whether or not the patient is in need of a shock or other intervention or assistance. Patient physiological parameters may also optionally include the patient's medical history, event history and so on. Examples of such parameters include the above-described electrodes to detect the ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring devices 180, 581 may include one or more sensors or transducers configured to acquire patient physiological signals. Examples of such sensors and transducers include one or more electrodes to detect ECG signals, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter reflects a trend that can be detected in a monitored physiological parameter of the patient 82, 582. Such a trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of the patient 82, 582 will know about a condition that is either not improving or deteriorating.

Patient state parameters include recorded aspects of the patient 582, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed of the patient can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WMS made according to embodiments may thus include a motion detector. In embodiments, a motion detector can be implemented within the outside monitoring device 180 or within the internal monitoring device 581. A motion detector of a WMS according to embodiments can be configured to detect a motion event. A motion event can be defined as is convenient, for example a change in posture or motion from a baseline posture or motion, etc. In such cases, a sensed patient parameter is motion. Such a motion detector can be made in many ways as is known in the art, for example by using an accelerometer and so on. In this example, a motion detector 587 is implemented within the monitoring device 581.

System parameters of a WMS can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if the monitoring device 180 or 581 includes a GPS location sensor as per the above, and if it is presumed or sensed that the patient is wearing the WMS.

The unit 500 includes a therapy delivery port 510 and a sensor port 519 in the housing 501. In contrast, in FIG. 4 these ports are located at the hub 446.

In FIG. 5, the therapy delivery port 510 can be a socket in the housing 501, or other equivalent structure. The therapy delivery port 510 includes electrical nodes 514, 518. Therapy electrodes 504, 508 are shown, which can be as the therapy electrodes 104, 108. Leads of the therapy electrodes 504, 508, such as the leads 105 of FIG. 1, can be plugged into the therapy delivery port 510, so as to make electrical contact with the nodes 514, 518, respectively. It is also possible that the therapy electrodes 504, 508 are connected continuously to the therapy delivery port 510, instead. Either way, the therapy delivery port 510 can be used for guiding, via electrodes, to the wearer at least some of the electrical charge that has been stored in an energy storage module 550 that is described more fully later in this document. When thus guided, the electric charge will cause the shock 111 to be delivered.

The sensor port 519 is also in the housing 501, and is also sometimes known as an ECG port. The sensor port 519 can be adapted for plugging in the leads of ECG sensing electrodes 509. The ECG sensing electrodes 509 can be as the ECG sensing electrodes 209. These ECG sensing electrodes 209, 509 can be configured to sense ECG signals of the ambulatory patient 82 along one or more channels. The ECG sensing electrodes 509 in this example are distinct from the therapy electrodes 504, 508. It is also possible that the sensing electrodes 509 can be connected continuously to the sensor port 519, instead. The electrodes 509 can be types of transducers that can help sense an ECG signal of the patient, e.g. a 12-lead signal, or a signal from a different number of leads, especially if they make good electrical contact with the body of the patient and in particular with the skin of the patient. As with the therapy electrodes 504, 508, the support structure can be configured to be worn by the patient 582 so as to maintain the sensing electrodes 509 on a body of the patient 582. For example, the sensing electrodes 509 can be attached to the inside of the support structure 170 for making good electrical contact with the patient, similarly with the therapy electrodes 504, 508.

Optionally a WMS according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel. As such, it will not flow too far away from the location it is released. The fluid can be used for both the therapy electrodes 504, 508, and for the sensing electrodes 509.

The fluid may be initially stored in a fluid reservoir, not shown in FIG. 5. Such a fluid reservoir can be coupled to the support structure. In addition, a WMS according to embodiments further includes a fluid deploying mechanism 574. The fluid deploying mechanism 574 can be configured to cause at least some of the fluid to be released from the reservoir, and be deployed near one or both of the patient body locations to which the therapy electrodes 504, 508 are configured to be attached to the patient's body. In some embodiments, the fluid deploying mechanism 574 is activated prior to the electrical discharge responsive to receiving an activation signal AS from the processor 530, which is described more fully later in this document.

In some embodiments, the unit 500 also includes a measurement circuit 520, as one or more of its modules working together with its sensors and/or transducers. The measurement circuit 520 senses one or more electrical physiological signals of the patient from the sensor port 519, if provided. Even if the unit 500 lacks a sensor port, the measurement circuit 520 may optionally obtain physiological signals through the nodes 514, 518 instead, when the therapy electrodes 504, 508 are attached to the patient. In these cases, the input reflects an ECG measurement. The patient parameter can be an ECG, which can be sensed as a voltage difference between electrodes 504, 508. In addition, the patient parameter can be an impedance (IMP. or Z), which can be sensed between the electrodes 504, 508 and/or between the connections of the sensor port 519 considered pairwise as channels. Sensing the impedance can be useful for detecting, among other things, whether these electrodes 504, 508 and/or the sensing electrodes 509 are not making good electrical contact with the patient's body at the time. These patient physiological signals may be sensed when available. The measurement circuit 520 can then render or generate information about them as inputs, data, other signals, etc. As such, the measurement circuit 520 can be configured to render a patient input responsive to a patient parameter sensed by a sensor. In some embodiments, the measurement circuit 520 can be configured to render a patient input, such as values of an ECG signal, responsive to the ECG signal sensed by the ECG sensing electrodes 509. More strictly speaking, the information rendered by the measurement circuit 520 is output from it, but this information can be called an input because it is received as an input by a subsequent stage, device or functionality.

The unit 500 also includes a processor 530. The processor 530 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

In embodiments, the processor 530 performs more tasks and the measurement circuit 520 performs fewer tasks. Either way, in embodiments one of the measurement circuit 520 and the processor 530 samples the sensed ECG signals to produce a sets of ECG values. The sampling can be performed, for instance, by an analog to digital converter (ADC), which provides the desired numerical ECG values for further processing. In some of these embodiments, the processor 530 further controls and may adjust the sampling rate.

The processor 530 may include, or have access to, a non-transitory storage medium, such as a memory 538 that is described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

The processor 530 can be considered to have a number of modules. One such module can be a detection module 532. The detection module 532 can include a Ventricular Fibrillation (VF) detector. The patient's sensed ECG from measurement circuit 520, which can be available as inputs, data that reflect values, or values of other signals, may be used by the VF detector to determine whether the patient is experiencing VF. Detecting VF is useful, because VF typically results in SCA. The detection module 532 can also include a Ventricular Tachycardia (VT) detector for detecting VT, and so on.

Another such module in processor 530 can be an advice module 534, which generates advice for what to do. The advice can be based on outputs of the detection module 532. There can be many types of advice according to embodiments. In some embodiments, the advice is a shock/no shock determination that processor 530 can make, for example via advice module 534. The shock/no shock determination can be made by executing a stored Shock Advisory Algorithm. A Shock Advisory Algorithm can make a shock/no shock determination from one or more ECG signals that are sensed according to embodiments, and determine whether or not a shock criterion is met. The determination can be made from a rhythm analysis of the sensed ECG signal or otherwise. For example, there can be shock decisions for VF, VT, etc.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in published US patent application No. US 2019/0030351 A1, and No. US 2019/0030352 A1, and which are incorporated herein by reference.

The processor 530 can include additional modules, such as other module 536, for other functions. In addition, if the internal monitoring device 581 is indeed provided, the processor 530 may receive its inputs, etc.

The unit 500 optionally further includes a memory 538, which can work together with the processor 530. The memory 538 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. The memory 538 is thus a non-transitory storage medium. The memory 538, if provided, can include programs for the processor 530, which the processor 530 may be able to read and execute. More particularly, the programs can include sets of instructions in the form of code, which the processor 530 may be able to execute upon reading. Executing is performed by physical manipulations of physical quantities, and may result in functions, operations, processes, acts, actions and/or methods to be performed, and/or the processor 530 to cause other devices or components or blocks to perform such functions, operations, processes, acts, actions and/or methods. The programs can be operational for the inherent needs of the processor 530, and can also include protocols and ways that decisions can be made by the advice module 534. In addition, the memory 538 can store prompts for the user 582, if this user is a local rescuer. Moreover, the memory 538 can store data. This data can include patient data, system data and environmental data, for example as learned by the internal monitoring device 581 and the outside monitoring device 180. The data can be stored in the memory 538 before it is transmitted out of the unit 500, or be stored there after it is received by the unit 500.

The unit 500 can optionally include a communication module 590, for establishing one or more wired or wireless communication links with other devices of other entities, such as a remote assistance center, Emergency Medical Services (EMS), and so on. The communication module 590 can be in the unit or not. The communication links can be used to transfer data and commands to an other device distinct from the unit 100. The data may be patient data, event information, therapy attempted, CPR performance, system data, environmental data, and so on. For example, the communication module 590 may transmit wirelessly, e.g. on a daily basis, heart rate, respiratory rate, and other vital signs data to a server accessible over the internet, for instance as described in US 20140043149. Or, this data may be sent to a base station 149 (seen in FIG. 1) at the home of the patient 82, either wirelessly or by direct electrical connection. In that case, the other device is the base station 149. For instance, the base station 149 may be combined with a battery recharger for the battery 440 (seen in FIG. 4), and/or a portion of the memory 538 can be on the battery 440. The communication module can be in the battery 440 to push the data to the base station 149, or in the base station 149 to pull the data from the battery 440. Then the base station 149 may transmit the data to the remote assistance center. This data can be analyzed directly by the patient's physician and can also be analyzed automatically by algorithms designed to detect a developing illness and then notify medical personnel via text, email, phone, etc. The module 590 may also include such interconnected sub-components as may be deemed necessary by a person skilled in the art, for example an antenna, portions of a processor, supporting electronics, outlet for a telephone or a network cable, etc.

The unit 500 may also include a power source 540, which is configured to provide electrical charge in the form of a current. To enable portability of the unit 500, the power source 540 typically includes a battery. Such a battery is typically implemented as a battery pack, which can be rechargeable or not. Sometimes a combination is used of rechargeable and non-rechargeable battery packs. An example of a rechargeable battery 540 was a battery 440 of FIG. 4. Other embodiments of the power source 540 can include an AC power override, for where AC power will be available, an energy-storing capacitor, and so on. Appropriate components may be included to provide for charging or replacing the power source 540. In some embodiments, the power source 540 is controlled and/or monitored by the processor 530.

The unit 500 may additionally include an energy storage module 550. The energy storage module 550 can be coupled to receive the electrical charge provided by the power source 540. The energy storage module 550 can be configured to store the electrical charge received by the power source 540. As such, the energy storage module 550 is where some electrical energy can be stored temporarily in the form of an electrical charge, when preparing it for discharge to administer a shock. In embodiments, the module 550 can be charged from the power source 540 to the desired amount of energy, for instance as controlled by the processor 530. In typical implementations, the module 550 includes a capacitor 552, which can be a single capacitor or a system of capacitors, and so on. In some embodiments, the energy storage module 550 includes a device that exhibits high power density, such as an ultracapacitor. As described above, the capacitor 552 can store the energy in the form of an electrical charge, for delivering to the patient.

As mentioned above, the patient is typically shocked when the shock criterion is met. In particular, in some embodiments the processor 530 is configured to determine from the patient input whether or not a shock criterion is met, and cause, responsive to the shock criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82. Delivering the electrical charge is also known as discharging and shocking the patient.

For causing the discharge, the unit 500 moreover includes a discharge circuit 555. When the decision is to shock, the processor 530 can be configured to control the discharge circuit 555 to discharge through the patient at least some of all of the electrical charge stored in the energy storage module 550, especially in a desired waveform. When the decision is to merely pace, i.e., to deliver pacing pulses, the processor 530 can be configured to cause control the discharge circuit 555 to discharge through the patient at least some of the electrical charge provided by the power source 540. Since pacing requires lesser charge and/or energy than a defibrillation shock, in some embodiments pacing wiring 541 is provided from the power source 540 to the discharge circuit 555. The pacing wiring 541 is shown as two wires that bypass the energy storage module 550, and only go through a current-supplying circuit 558. As such, the energy for the pacing is provided by the power source 540 either via the pacing wiring 541, or through the energy storage module 550. And, in some embodiments where only a pacer is provided, the energy storage module 550 may not be needed if enough pacing current can be provided from the power source 540. Either way, discharging can be to the nodes 514, 518, and from there to the therapy electrodes 504, 508, so as to cause a shock to be delivered to the patient. The circuit 555 can include one or more switches 557. The switches 557 can be made in a number of ways, such as by an H-bridge, and so on. In some embodiments, different ones of the switches 557 may be used for a discharge where a defibrillation shock is caused to be delivered, than for a discharge where the much weaker pacing pulses are caused to be delivered. The circuit 555 could also be thus controlled via the processor 530, and/or the user interface 580.

The pacing capability can be implemented in a number of ways. ECG sensing may be done in the processor, as mentioned elsewhere in this document, or separately, for demand or synchronous pacing. In some embodiments, however, pacing can be asynchronous. Pacing can be software controlled, e.g., by managing the defibrillation path, or a separate pacing therapy circuit (not shown) could be included, which can receive the ECG sensing, via the circuit 520 or otherwise.

A time waveform of the discharge may be controlled by thus controlling discharge circuit 555. The amount of energy of the discharge can be controlled by how much energy storage module has been charged, and also by how long the discharge circuit 555 is controlled to remain open.

The unit 500 can optionally include other components.

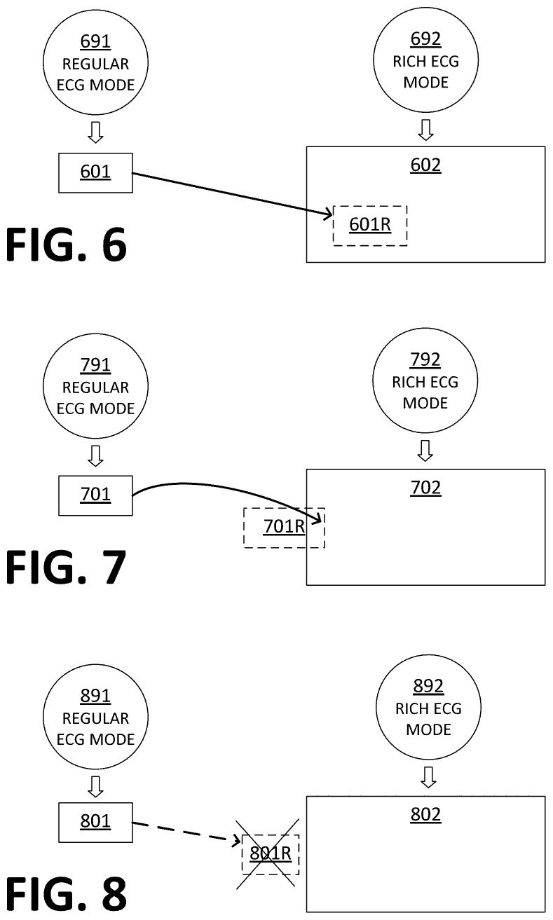

Referring now to FIG. 6, a WMS according to embodiments may operate in at least two modes of sensing ECG values, and recording them by storing them in the memory 538. The first mode 691, which can also be called a regular mode and a regular ECG mode, can be used to sense and record a first set 601 of ECG values. The first set 601 can be used to determine whether or not the patient 82 needs to be defibrillated, for the WCD operation of the WMS. Typically the ECG values of only one channel are needed for the first set 601. Even where the ECG signals of more than one channels are available and sampled, the channel with the best ECG values is typically selected and recorded as the first set 601, while the ECG values of the other channels are ignored. This selection process typically depends on criteria for deciding which channel provides the ECG signal that is the best to analyze. These criteria include deciding which channel is the freest of noise, which has the best signal-to-noise ratio, and so on.

The second mode 692, which can also be called the rich mode or rich ECG mode, can be used to sense and record a second set 602 of ECG values. This can be accomplished in a number of ways, as described later in this document. The rich ECG mode can be implemented for the long-term characterization of the heart. It can provide a more detailed characterization of the heart, which has additional advantages. The second set 602 can be recorded with further annotations, such as determinations made on the fly by the processor 530, and so on. Such determinations may include a record of date and time, patient recorded inputs, noise determinations, and so on. Of course, in embodiments, ECG data from the rich ECG mode can also be used to determine whether or not the patient 82 needs to be defibrillated.

FIG. 6 further illustrates an embodiment where the second set 602 resulting from a rich ECG mode 692 includes the entire first set 601 resulting from a regular ECG mode 691. This inclusion is depicted by showing a replica 601R of the first set 601 entirely within the second set 602. In other words, the second set 602 is a superset of the first set 601, and the rich ECG mode state 692 is implemented by simply adding capabilities to the regular mode 691.

FIG. 7 illustrates an embodiment where a second set 702 of ECG values resulting from a rich mode 792 includes only a part of a first set 701 of ECG values resulting from a regular ECG mode 791. This partial inclusion is depicted by showing a replica 701R of the first set 701 only partly within the second set 702.

FIG. 8 illustrates an embodiment where a second set 802 of ECG values resulting from a rich mode 892 includes none of a first set 801 of ECG values resulting from a regular ECG mode 891. This non-inclusion is depicted by showing a replica 801R of the first set 801 entirely outside the second set 802.

In FIGS. 6-8, the second sets were shown as larger than their corresponding first sets. That is because more ECG data can be collected in the rich mode than in the regular mode. This is now shown in more detail.

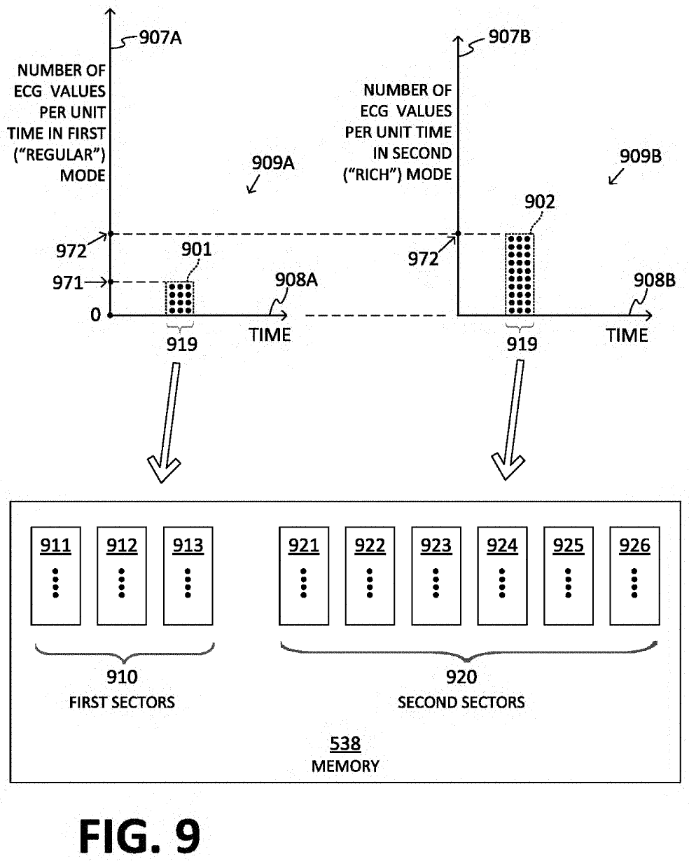

FIG. 9 shows a first time diagram 909A and a second time diagram 909B. The first time diagram 909A has a horizontal time axis 908A, along which a time interval 919 can be shifted. The first time diagram 909A also has a vertical axis 907A, for counting the number of ECG values recorded per unit time in a first mode of operation, which can be the regular mode. The second time diagram 909B has a horizontal time axis 908B, along which the time interval 919 can be shifted. In this example where the horizontal time axis 908B has the same scale as the horizontal time axis 908A. The horizontal time axis 908B is aligned with the horizontal time axis 908A, as indicated by a dashed line. The second time diagram 909B also has a vertical axis 907B, for counting the number of ECG values recorded per unit time in a second mode of operation, which can be the rich mode.

In the first time diagram 909A, a first set 901 of ECG values is shown. The individual ECG values themselves are depicted as small black dots, and the first set 901 is shown as a rectangle that surrounds them. The representation with the rectangle is intended to visually convey their total number, for easy comparison with the numbers of other sets. This first set 901 is of ECG values that are sampled over the time interval 919. This first set 901 has a first number 971 of ECG values per unit time, as measured on the vertical axis 907A.

Similarly, in the second time diagram 909B, a second set 902 of ECG values is shown. The individual ECG values themselves are depicted as small black dots, and the second set 902 is shown as a rectangle that surrounds them. This second set 902 is of ECG values that are sampled over the time interval 919. This second set 902 has a second number 972 of ECG values per unit time, as measured on the vertical axis 907B.

In embodiments, the second number 972 of ECG values per unit time is larger than the first number 971 of ECG values per unit time. The comparison is illustrated by showing the second number 972 also on the vertical axis 907A of the first time diagram 909A. The second number 972 can be at least twice as large as the first number 971, or much larger, for instance at least 5 times, at least 10 times, and so on.

In embodiments, therefore, the processor 530 can be further configured to store in the memory 538 a) the first set 901 of ECG values produced by sampling the sensed ECG signals, and b) a second set 902 of ECG values produced by sampling the sensed ECG signals. In the example of FIG. 9, the storing is depicted in the lower portion of the drawing, which shows a detail of the memory 538 as may be implemented in some embodiments. The memory 538 has a set 910 of first sectors 911, 912, 913, and a set 920 of second sectors 921, 922, 923, 924, 925, 926. As can be seen, portions of the first set 901 of ECG values are stored in the first sectors 911, 912, 913 of the memory 538, while portions of the second set 902 of ECG values are stored in the second sectors 921, 922, 923, 924, 925, 926 of the memory 538. More sectors of the memory 538 are used for the second set 902 than for the first set 901, because there are more ECG values to store, for the same time of sampling.

As mentioned above, in embodiments the processor 530 can be further configured to determine whether or not the shock criterion is met from at least one of the first set 901 of ECG values and the second set 902 of ECG values. For instance the processor 530 can choose ECG values available at the time, depending on which mode is being used.

Referring to FIG. 1, the memory 538 is also repeated outside the unit 100. The memory 538 stores a first set 101 of ECG values recorded during a regular mode, which is also called a first set and a regular set of ECG values. The memory 538 also stores a second set 102 of ECG values recorded during a rich mode, which is also called a second set and a rich set of ECG values.

Returning to FIG. 5, in embodiments the communication module 590 is configured to communicate the first set 101 of ECG values and the second set 102 of ECG values to an other device. The communication can be after the processor 530 has thus stored them in the memory 538, for instance at least 20 minutes, and possibly hours, until thus downloaded. This is possible by maintaining the first set of ECG values and the second set of ECG values stored in the memory 538 during that time. Or, they can be shifted around to different portions of the memory 538. After thus communicating the ECG values to the other device, the memory 538 can be freed for storing additional ECG data, for instance by overwriting, and so on.

Returning to FIG. 9, the first set 901 and the second set 902 are purposely shown in separate time diagrams 909A, 909B so as to not require a time relationship between them. in fact, there is a number of such possible relationships.

In some embodiments, a WMS can operate in the regular mode and in the rich mode concurrently. For instance, at a certain time moment, the processor 530 can be configured to thus store the first set 901 of ECG values, and to concurrently thus store the second set 902 of ECG values. At that certain time moment, the first set 901 of ECG values is being thus stored, and also the second set 902 of ECG values is being thus stored. In such embodiments, the second sectors 921, 922, 923, 924, 925, 926 might not be interspersed among the first sectors 911, 912, 913; rather, the first sectors might be grouped by themselves, and the second sectors might be grouped by themselves.

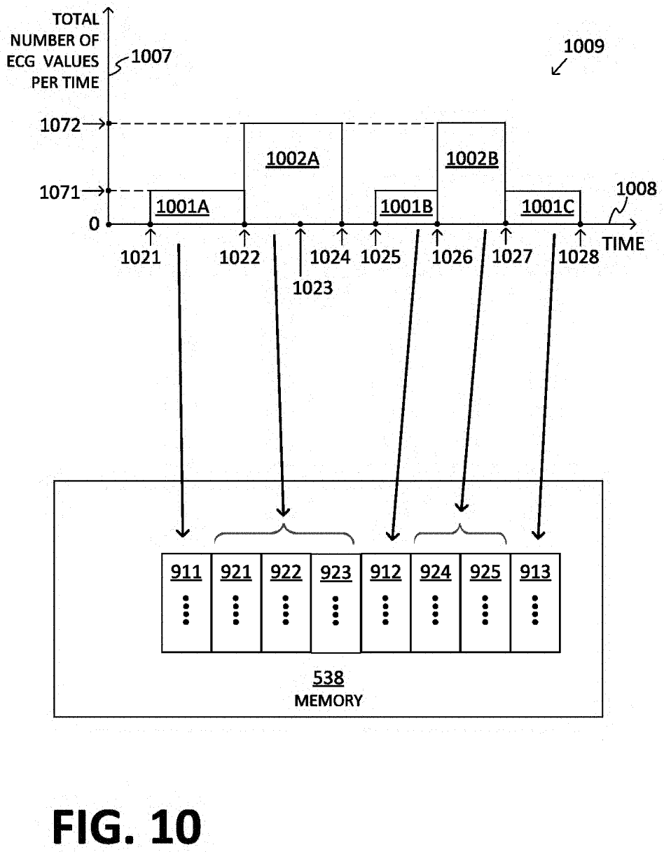

FIG. 10 shows how, in some embodiments, a WMS can operate either in the regular mode or in the rich mode, but not both concurrently. A time diagram 1009 has a horizontal time axis 1008 and a vertical axis 1007. The vertical axis 1007 is for counting the total number of ECG values recorded per unit time, as it that number is different during different modes of operation. The modes of operation change at time moments 1021, 1022, 1024, 1025, 1026, 1027, 1028. No ECG values are shown recorded before the time moment 1021, or after time moment 1028, and that is done only artificially, since those times are not of interest for the present description.

Portions 1001A, 1001B, 1001C of a first set of ECG values are recorded in a regular mode, during the intervals between the time moments 1021-1022, 1025-1026 and 1027-1028 respectively. During those intervals, the total number of recorded ECG values per unit time is 1071, as seen on the vertical axis 1007.

In addition, portions 1002A, 1002B of a second set of ECG values are recorded in a rich mode, during the intervals between the time moments 1022-1024 and 1026-1027 respectively. During those intervals, the total number of recorded ECG values per unit time is 1072.

It will be observed that there can be intervals when no ECG values are recorded, between operations of the regular mode and the rich mode. One such interval is between the time moments 1024-1025; this may happen for a number of reasons. One such reason is during defibrillation but, in that case, one may prefer to restart quickly with the rich ECG mode, such as a 12-lead ECG, instead of what is shown in the example of FIG. 10. This permits obtaining a fuller picture of the heart as it hopefully restarts, electrical artifacts, and so on. In embodiments, therefore, the processor is further configured to: store in the memory additional ECG values of the second set, responsive to causing the at least some of the stored electrical charge to be thus discharged, within 10 sec from thus causing, or even faster, such as 5 sec, 3 sec and so on.

The example of FIG. 10 illustrates operation in either regular mode or rich mode but not both concurrently. For instance, at a certain time moment, the processor 530 is configured to thus store either the first set of ECG values, made from the portions 1001A, 1001B, 1001C, or the second set of ECG values, made from the portions 1002A, 1002B, but not both. At that certain time moment, either the first set of ECG values is being thus stored, or the second set of ECG values is being thus stored, but not both. For instance, at the sample time moment 1023, the processor is recording only the second set.

It should be noted that, in FIG. 10, the time differences between the shown time moments are not necessarily to scale! Indeed, in the regular mode only time slivers of the first set may be recorded and retained, and only if it is detected that the patient 82 is having a heart-related episode. For instance, it is possible that no such data may be retained if the patient 82 has a full night's restful sleep with no episodes. On the other hand, in the rich mode, ECG values may be recorded for long stretches of time, regardless of whether the patient is having a heart-related episode. In fact, rich ECG data may be obtained while the patient is having a full night's sleep.

In the example of FIG. 10, the storing is depicted in the lower portion of the drawing, which shows a detail of how the memory 538 may become mapped in this example. The portions 1001A, 1001B, 1001C are stored in the first sectors 911, 912, 913, respectively. The portion 1002A is stored in the second sectors 921, 922, 923, and the portion 1002B is stored in the second sectors 924, 925. This is an example of where at least some of the second sectors are interspersed among the first sectors, which can take place because they store ECG values along a time continuum.

In some embodiments, a WMS can operate routinely in the regular mode, and occasionally also in the rich mode in addition to the regular mode. And, In some embodiments, a WMS can use a combination of modes.

From the above, situations can be considered where the rich mode is turned on and off, regardless of whether the regular mode is accordingly affected. The regular mode would be affected or not based on what was described in FIGS. 6-10. Examples of turning on and off the rich mode are now described.

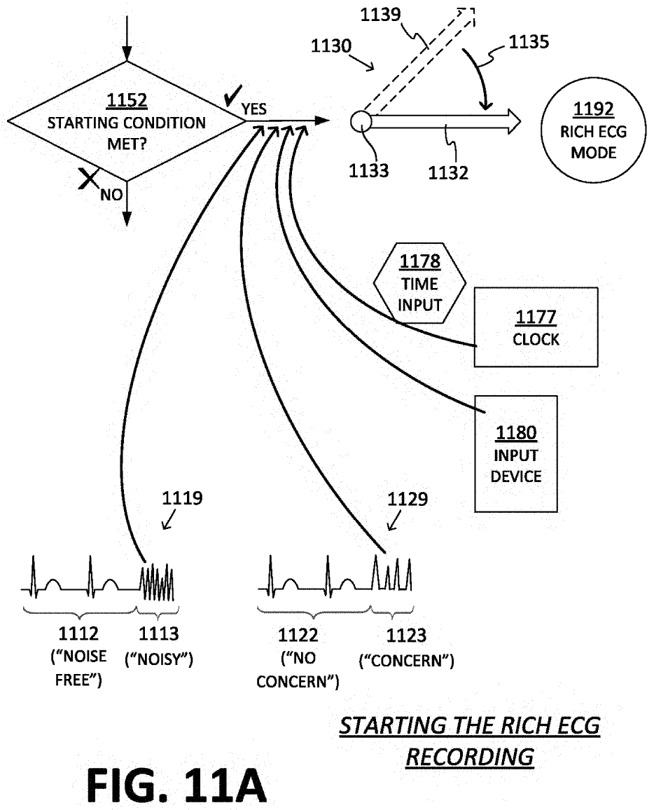

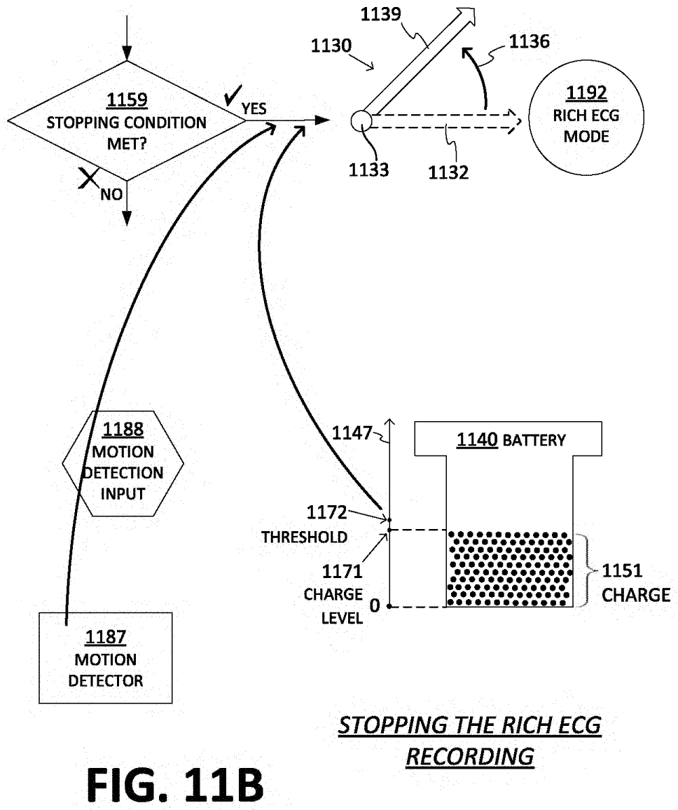

FIG. 11A shows a rich ECG mode state 1192, as it might be drawn if it were a part of a larger state diagram. The rich ECG mode state 1192 may be implemented explicitly as part of a state machine of the processor 530, or of other components, or implemented implicitly, and so on. Consistently with the above, the rich ECG mode state 1192 in this example is shown independently of whether the regular mode is on or off.

In the example of FIG. 11A, a state arrow 1130 is at an on-position 1132, which shows that the rich ECG mode state 1192 is being switched on. The state arrow 1130 can rotate around a point 1133 between the on-position 1132 and an off-position 1139. The off-position 1139 does not necessarily speak to whether or not the regular mode is turned on, or ECG data is not being sampled at all. The rich ECG mode state 1192 is turned on or switched on when, according to an arrow 1135, the state arrow 1130 rotates from the off-position 1139 to the on-position 1132.

FIG. 11A also shows a decision diamond 1152 according to embodiments. The decision diamond 1152 could be part of a flowchart describing a method, algorithm implemented by a program, and so on. The decision diamond 1152 may be reached and caused to be executed while the state arrow 1130 is in the off-position 1139, or even when it is in the on-position 1132. The latter can be, for example when independent events can cause execution to reach the decision diamond 1152. In some embodiments, the decision diamond 1152 is not executed if, when it is reached, the state arrow 1130 is already in the on-position 1132.

In some embodiments, according to the decision diamond 1152, the processor 530 is further configured to detect whether or not a starting condition is met. In such embodiments, the second set of ECG values starts being thus recorded responsive to the starting condition being met. In this example, if the starting condition is met then, according to a YES branch of the decision diamond 1152, the operation of the arrow 1135 can be performed. But if the starting condition is not met then, according to a NO branch of the decision diamond 1152, execution can proceed to another operation (not shown).

The starting condition of the decision diamond 1152 can be implemented by a number of events, which may even be independent of each other. Whether or not the starting condition applies can be checked in a number of ways. For instance, such events might register with the processor 530 as interrupts, or as values of variables that are routinely checked by the processor 530. Examples of such events are now described.

In some embodiments, the processor 530 is further configured to detect, while storing the first set 101, 601, 701, 801, 901 of ECG values, noise in the one or more channels that is above a noise threshold. The noise threshold can be set by the number of the needed readable channels available in the regular mode. At least one is needed, with a signal to noise ratio (SNR) that exceeds a certain SNR threshold. The noise threshold can be set per how many channels must be available for specific SNR thresholds. In such embodiments, the starting condition can be met responsive to the detected noise. In the example of FIG. 11A, a sample ECG waveform 1119 is shown, as it may have been sampled from the first set 101, 601, 701, 801, 901 of ECG values, and from the only noise-free channel of the regular mode. An initial portion 1112 of the sample ECG waveform 1119 is read clearly, and is characterized as “NOISE FREE”. Here the attentive reader will notice that the initial portion 1112 also shows a healthy heart rhythm, but that is only in this example; it could be an unhealthy heart rhythm, and still be read clearly and characterized as “NOISE FREE”. In a subsequent portion 1113 of the sample ECG waveform 1119, noise is detected, and that portion 1113 can be characterized as “NOISY”. This can cause the starting condition to be met, as indicated by an arrow from the portion 1113 to the YES branch of the decision diamond 1152. This could start the rich mode, if more channels will be provided than in the regular mode. As such, in the rich mode, the algorithm has an even better chance to detect whether or not the shock criterion is met. Moreover, there is a better chance of capturing and diagnosing intermittent atrial fibrillation and VT storm. Operationally this has the potential benefit of reducing the false alarm and inappropriate shock rates.

In some embodiments, the processor 530 is further configured to detect an arrhythmia from the first set 101, 601, 701, 801, 901 of ECG values. In such embodiments, the starting condition can be met responsive to the arrhythmia being detected. In the example of FIG. 11A, a sample ECG waveform 1129 is shown, as it may have been sampled from the first set 101, 601, 701, 801, 901 of ECG values. An initial portion 1122 of the sample ECG waveform 1129 can be as a normal rhythm and can therefore be characterized as “NO CONCERN”. However, a subsequent portion 1123 of the sample ECG waveform 1129 can be detected as an arrhythmia, and can therefore be characterized as “CONCERN”. This can cause the starting condition to be met, as indicated by an arrow from the portion 1123 to the YES branch of the decision diamond 1152. This would start the rich mode, which provides more ECG values per time, and can help analyze better the portion 1123. As such, in the rich mode, the algorithm has a better chance to distinguish atrial (non-shockable) tachycardias from ventricular (shockable) tachycardias. Moreover, such intermittent use of the rich mode permits economizing on expended electrical power.

In some embodiments, a WMS further includes an input device 1180 that can be configured to be actuated by the ambulatory patient 82. The input device 1180 can be part of the user interface 580, such as a physical button, a button in the UI of a screen, a microphone with processing to detect voice commands, etc. In such embodiments, the starting condition can be met responsive to the input device 1180 being actuated by the patient 82, as indicated by an arrow from the input device 1180 to the YES branch of the decision diamond 1152. For instance, the patient 82 may have been instructed to start the rich mode if they are not feeling well, or if they are feeling different than usual.

In some embodiments, a WMS further includes a clock 1177 that can be configured to render a time input 1178. The clock 1177 can be implemented by the processor 530 internally, or by receiving the time from a network, and so on. In such embodiments, the starting condition can be met responsive to the time input 1178 meeting a suitability criterion, as indicated by an arrow from the clock 1177 to the YES branch of the decision diamond 1152. The suitability criterion may include that the time is a certain time of the night at a location of the patient. This way a 4, or even 6-hour rich ECG recording may be obtained.

FIG. 11B shows the rich ECG mode state 1192, similarly with FIG. 11A. The state arrow 1130 shows the rich ECG mode state 1192 being switched off, by the state arrow 1130 rotating from the on-position 1132 to the off-position 1139 according to an arrow 1136.

FIG. 11B also shows a decision diamond 1159 according to embodiments. The decision diamond 1159 could be part of a flowchart describing a method, algorithm implemented by a program, and so on. The decision diamond 1159 may be reached and caused to be executed while the state arrow 1130 is in the on-position 1132, or even when it is in the off-position 1139. The latter can be, for example when independent events can cause execution to reach the decision diamond 1159. In some embodiments, the decision diamond 1159 is not executed if, when it is reached, the state arrow 1130 is already in the off-position 1139.

In some embodiments, according to the decision diamond 1159, the processor 530 is further configured to detect whether or not a stopping condition is met. In such embodiments, the second set of ECG values stops being thus recorded responsive to the stopping condition being met. In this example, if the stopping condition is met then, according to a YES branch of the decision diamond 1159, the operation of the arrow 1136 can be performed. But if the stopping condition is not met then, according to a NO branch of the decision diamond 1159, execution can proceed to another operation (not shown).

The stopping condition of the decision diamond 1159 can be implemented by a number of events, which may even be independent of each other, similarly with what was described above with reference to the decision diamond 1152. Examples of such events are now described.

In some embodiments, a WMS further includes a motion detector 1187 that can be configured to render a motion detection input 1188. In such embodiments, the stopping condition can be met responsive to the motion detection input 1188 meeting an unrest criterion, as indicated by an arrow from the motion detector 1187 to the YES branch of the decision diamond 1159. The unrest criterion might be crafted such that it indicates when the patient 82 is moving, momentarily or continuously, in which case it may be presumed that there will be electrical noise and therefore the resulting ECG data will not be useful for analysis.

For implementing embodiments, it may be recognized that the rich ECG mode will consume more energy than the regular EVG mode, in fact possibly much more energy per time.

A number of solutions to the additional energy requirement are presented in this document. One such solution is that, in some embodiments, a WMS further includes a battery 1140 that is configured to be inserted into the unit 100 so as to power the processor 530. The battery 1140, similarly with the battery 440 and the power source 540, can be configured to be inserted into the unit 100 so as to power the processor 530. The battery 1140 can be configured to store an electrical charge 1151, and to supply the stored electrical charge to the energy storage module 550. In such embodiments, the processor 530 can be further configured to input a charge level 1171 of the electrical charge 1151 stored in the battery 1140. In some instances, the charge level 1171 is given as a percentage, for example 100% for a fully recharged battery, and so on, all along a vertical axis 1147. In such embodiments, the stopping condition can be met responsive to the inputted charge level 1171 being below a threshold 1172, as indicated by an arrow from the threshold 1172 to the YES branch of the decision diamond 1159.

The threshold 1172 can be set according to projected needs and capabilities. For instance, it may be set so that the battery 1140 will have enough charge 1151 to provide for monitoring in the regular mode for 11 hours, plus for three shocks in the event that they are needed. The time margin can be different during the daytime if it is detected that the patient is not sleeping, or close to the morning while the patient is sleeping. For assisting these calculations, it may be useful to consider the following:
i) In the regular mode, there may be ECG data from one (1) channel that have been sampled at a first sampling rate. So, in one minute of the regular mode a certain amount of energy will be consumed, for capturing and storing the ECG data.
ii) In the rich mode, there may be 12 channels (×12) or even more, and the sampling rate could be double (×2) the first sampling rate, as will be shown later in this document. So, in one minute of the rich mode, the same amount of energy will be consumed as in 12×2=24 minutes of the regular mode, for capturing and storing the ECG data.
iii) The above computations for the differences in energy budgets may or may not be the only ones needed. For instance, even in the rich mode, for the task of determining whether the patient is having an episode, whether or not the shock criterion is met, and so on, it may be adequate to analyze only one channel.

A different solution can be to modify the WMS to be chargeable via line power by using a cable, in addition to the power provided by the power source 540. Of course, this is generally not desired because it may severely restrict the mobility of the patient 82, but this might not be a problem during the night, or while working at a desk.

As mentioned above, the rich mode may be implemented by recording more ECG data per time compared to the regular mode. This can be implemented in a number of ways, one of which is to increase the sampling rate relative to the regular mode, without even increasing the number of ECG sensing electrodes or channels. Examples are now described.

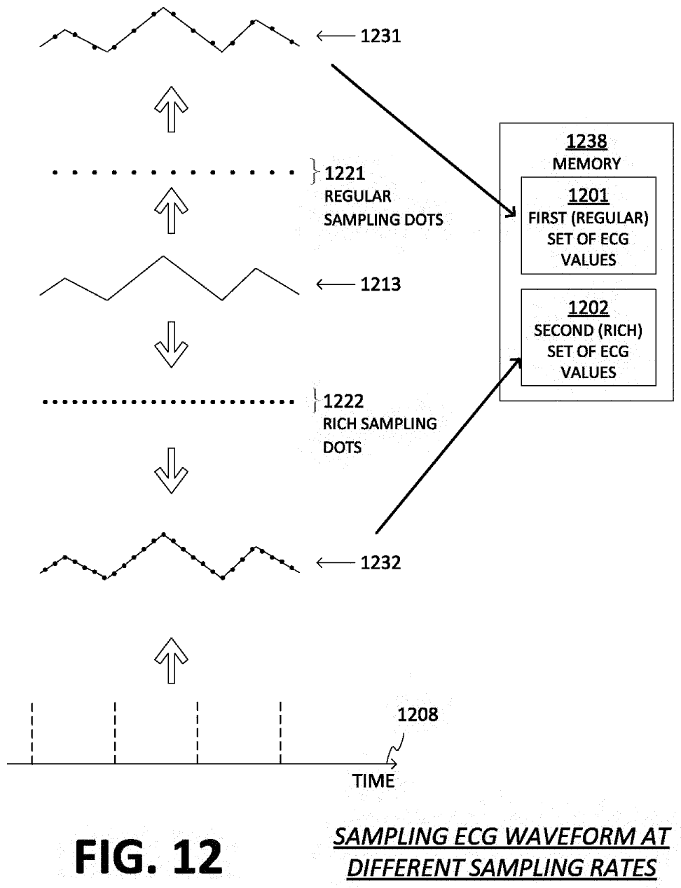

Referring now to FIG. 12, a time axis 1208 applies to all waveforms above it, as indicated by an upward pointing arrow from it. Dashed lines extend upwards from it, but not enough to actually reach all the other waveforms, to prevent from cluttering the drawing.

A sample sensed ECG signal 1213 is shown. Compared to previously shown ECG signal waveforms, the sensed ECG signal 1213 is “stretched out” horizontally, on a very slow-moving, high resolution time axis, and therefore includes very few up-down transitions, for purposes of the explanation in the example of FIG. 12.

In some embodiments, a first set 1201 of ECG values is produced by sampling the sensed ECG signal 1213 at a first sampling rate. Such a first sampling rate is depicted here conceptually by regular sampling dots 1221, which occur at periodic time intervals. A sampled waveform 1231 is produced from the sensed ECG signal 1213, after the regular sampling dots 1221 have been superimposed on it. Each such dot indicates an ECG value that is thus obtained.

In addition, a second set 1202 of ECG values is produced by sampling the sensed ECG signal 1213 at a second sampling rate. The second sampling rate can be at least 50% faster than the first sampling rate, twice as fast, and so on. The second sampling rate can be at least 740 ECG values per sec, for instance 1000 ECG values per sec. Such a second sampling rate is depicted here conceptually by rich sampling dots 1222, which occur at periodic time intervals. In this example, the rich sampling dots 1222 occur at twice the speed or frequency of the regular sampling dots 1221. A sampled waveform 1232 is produced from the sensed ECG signal 1213, after the rich sampling dots 1222 have been superimposed on it. Each such dot indicates an ECG value that is thus obtained.

The first set 1201 of ECG values and the second set 1202 of ECG values can be stored in a memory 1238 that can be as the memory 538. This drawing makes visually apparent that the ECG values captured in the rich mode are more numerous than those captured in the regular mode, as the sampled waveform 1232 has twice the dots that sampled waveform 1231 has, for the same amount of time. This makes the second set 1202 of ECG values more amenable for detailed study of the heart 85.

It will be appreciated that embodiments can be implemented with, say, four ECG sensing electrodes, such as the ECG sensing electrodes 209 of FIG. 2A. An example is now described.

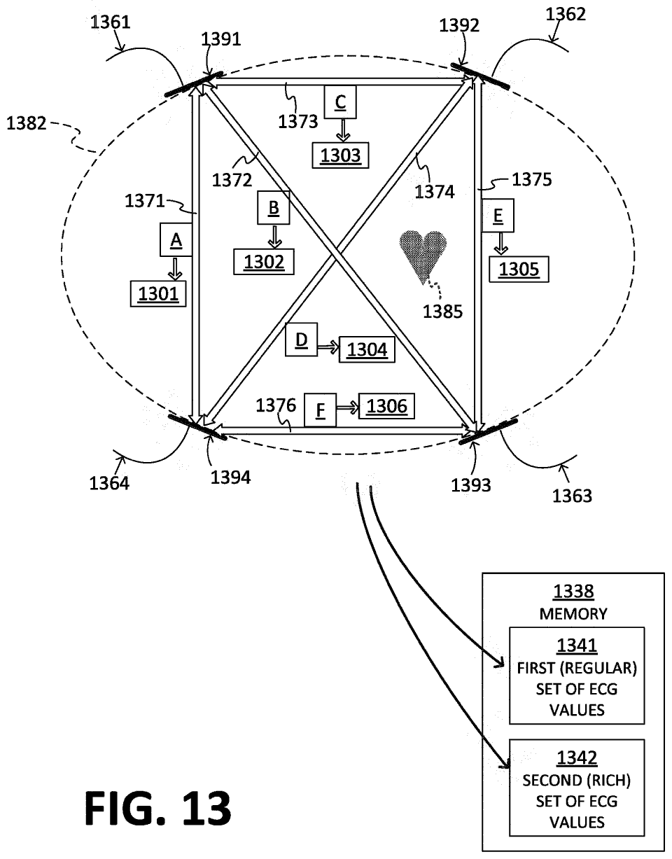

FIG. 13 is a diagram that illustrates conceptually how multiple ECG sensing electrodes may be used for sensing ECG signals along different channels in a WMS that implements a WCD according to embodiments, to collect ECG values in a regular mode and/or in a rich mode. As mentioned above, in the regular mode the ECG signals from a number of channels are available but, as only one is needed, the other ECG signals are typically ignored—not analyzed and definitely not stored. However, in the rich mode, the other ECG signals are typically not ignored.

In embodiments, the shock/no shock decision can be made from the patient's heart rate and/or the QRS width of the patient's ECG complexes in the patient's ECG signal. Other parameters may also be used, such as information from a patient impedance signal (Z), information from a motion detection signal (MDET) that may evidence a motion of the patient, and so on. Of course, it is desired to measure these parameters as accurately as possible.

ECG signals in a WCD system may include too much electrical noise to be useful. To ameliorate the problem, multiple ECG sensing electrodes 209 are provided, for presenting many options for the processor 530 to choose one, for the regular mode. These options are different channels for sensing the ECG signal, as described now in more detail.

FIG. 13 is a conceptual diagram for illustrating how multiple electrodes of a WCD system may be used for sensing ECG signals along the channels of different vectors according to embodiments. A section of a patient 1382 having a heart 1385 is shown. In FIG. 13, the patient 1382 is viewed from the top, the patient 1382 is facing downwards, and the plane of FIG. 13 intersects the patient 1382 at the torso of the patient.

Four ECG sensing electrodes 1391, 1392, 1393, 1394 are maintained on the torso of the patient 1382, and have respective wire leads 1361, 1362, 1363, 1364. It will be recognized that the electrodes 1391, 1392, 1393, 1394 surround the torso, similarly with the four ECG sensing electrodes 209 of FIG. 2A. The ECG electrical potentials that can be measured at the electrodes 1391, 1392, 1393, 1394 can have values E1, E2, E3, E4.

Any pair of these four ECG sensing electrodes 1391, 1392, 1393, 1394 defines a vector, which defines a channel, along which an ECG signal may be sensed and/or measured. As such, the four electrodes 1391, 1392, 1393, 1394 pairwise define six vectors 1371, 1372, 1373, 1374, 1375, 1376. FIG. 13 thus illustrates a multi-vector embodiment. Although four electrodes, and thus six vectors, are shown in the example of FIG. 13, other numbers can be implemented.

In FIG. 13 it will be understood that the electrodes 1391, 1392, 1393, 1394 are drawn as if they were on the same plane. This is done because simplicity of explanation is preferred but, strictly speaking, it is not necessarily the case. In fact, the electrodes 1391, 1392, 1393, 1394 might not always be on the same plane, in which case the vectors 1371, 1372, 1373, 1374, 1375, 1376 are not necessarily on the same plane, either.

These vectors 1371, 1372, 1373, 1374, 1375, 1376 define channels A, B, C, D, E, F respectively. ECG signals 1301, 1302, 1303, 1304, 1305, 1306 may thus be sensed and/or measured from the channels A, B, C, D, E, F, respectively, and in particular from the appropriate pairings of the wire leads 1361, 1362, 1363, 1364 for each channel. The ECG signals 1301, 1302, 1303, 1304, 1305, 1306 may be sensed concurrently or not.

The above-mentioned formalism gives or renders values of the ECG signal that is sensed between pairs of the electrodes. For instance, the ECG signal 1301 at channel A has a voltage E1−E2=E12.

In the example of FIG. 13, it is also possible to use a different formalism that produces ECG values, also known as ECG signal values, for each electrode by itself and at its location, not in a pair with another. This different formalism starts by imagining a point at a virtual position between the 4 electrodes 1391, 1392, 1393, 1394, somewhere within the torso of the patient 1382. (Such a point is not shown in FIG. 13.) An ECG voltage CM is ascribed to that point. That voltage CM is derived from a statistic of the voltages of at the four electrodes 1391, 1392, 1393, 1394. That statistic can be the average. The virtual position continuously changes its virtual position based on the voltages of the four electrodes 1391, 1392, 1393, 1394. Of course, there is no actual sensor for sensing the voltage at that point. Nevertheless, this different formalism further imagines a virtual main central terminal (MCT), not shown in FIG. 13, and which is what would be sensing that voltage CM.

In this different formalism, therefore, vectors are considered from each of the four electrodes 1391, 1392, 1393, 1394 to the MCT. Their values of their signals, therefore, are considered to be: E1C=E1−CM, E2C=E2−CM, E3C=E3−CM and E4C=E4−CM. In embodiments, the vectors are formed in software by selecting a pair of these signals and subtracting one from the other. So for example, E1C−E2C=(E1−CM)−(E2−CM)=E1−E2+(CM−CM)=E1−E2=E12.

Thus, having multiple channels A, B, C, D, E, F, a WCD may assess which one of them provides the best ECG signal for rhythm analysis and interpretation. Or, instead of just one channel, a WCD may determine that it can keep two or more but not all of the channels and use their ECG signals, for instance as described in U.S. Pat. No. 9,757,581.

FIG. 13 also shows a memory 1338, which can be as the memory 538. The memory 1338 stores a first set 1341 and a second set 1342 of ECG values. The first set 1341 may have been generated by the first sampling rate of FIG. 12, which is exemplified by the regular sampling dots 1221. The second set 1342 may have been generated by the second sampling rate of FIG. 12, which is exemplified by the rich sampling dots 1222. The second set 1342 has more ECG values than the first set 1341, even though the exact same number of ECG sensing electrodes is used.

Returning to FIG. 1, as mentioned above, the rich mode may be also by implemented by increasing the number of ECG sensing electrodes or channels compared to the regular mode. This can be implemented together with increasing the sampling rate, as seen above, or even without increasing the sampling rate. In some embodiments, the first set 101 of ECG values is produced by sampling the ECG signals that are sensed along at least one but no more than six of the one or more channels, but the second set 102 of ECG values is produced by sampling the ECG signals that are sensed concurrently along at least seven of the one or more channels. In some of these embodiments, the support structure 170 supports more than four ECG sensing electrodes, for instance six, seven, eight, ten, twelve, sixteen, and so on ECG sensing electrodes. An example is now described.

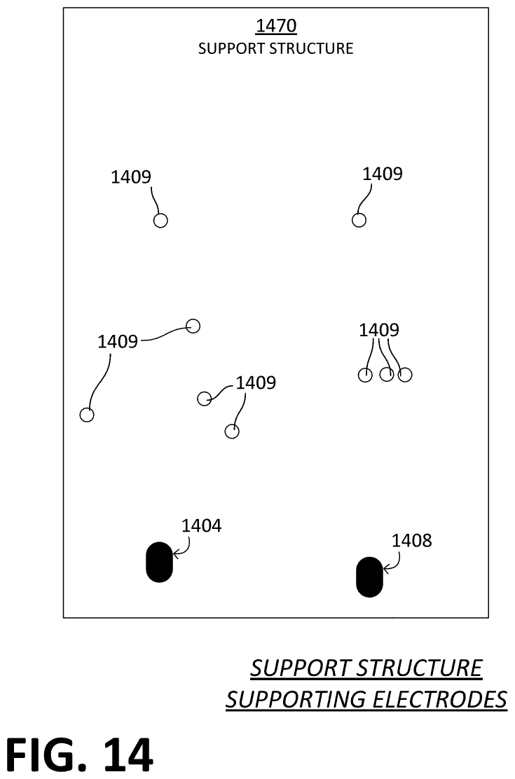

FIG. 14 is a diagram showing a support structure 1470. The support structure 1470 supports multiple ECG sensing electrodes 1409 in addition to defibrillation electrodes 1404, 1408. These ECG sensing electrodes 1409 may be placed at any desirable locations, and FIG. 14 does not speak as to the positions of these electrodes. For instance, a single ECG electrode high up on the back in addition to those of FIG. 13 may render four very useful vectors through the heart.

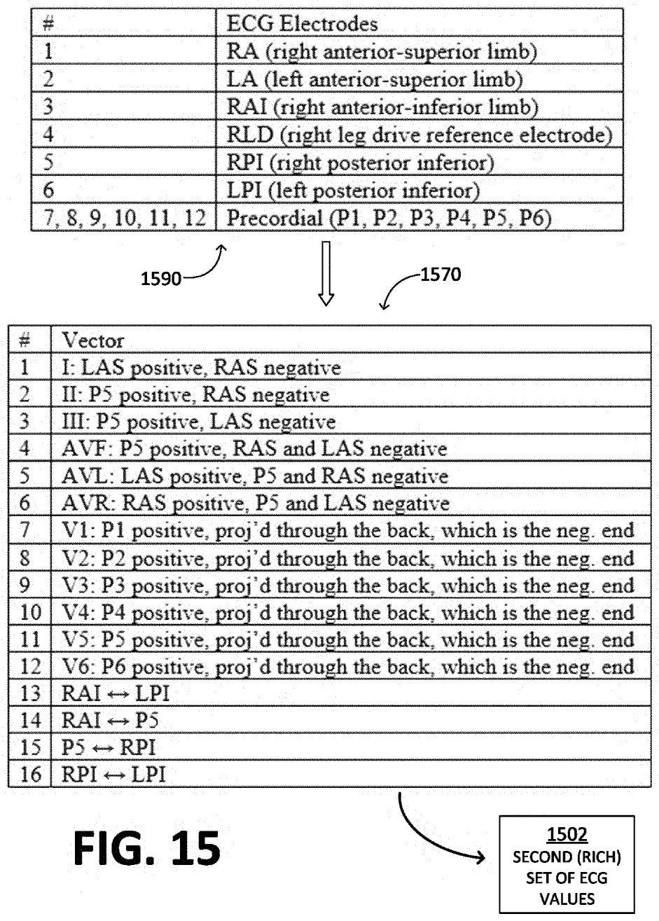

FIG. 15 shows a first table 1590 of a numbered list of locations of ECG sensing electrodes on a support structure of a WMS that implements a WCD according to embodiments. These locations are an example of locations possible for the support structure 1470 of FIG. 14. Of those, it will be appreciated that #4 RLD (“right leg drive”) is an electronics term, and does not refer to attaching anything to a leg of the patient.

FIG. 15 also shows a second table 1570 of a numbered list of potential vectors that may result from the ECG sensing electrodes of the table 1590, as indicated by an arrow. The ECG signals may be sensed from the respective channels of only the first 12 vectors of the table 1570, or all 16 vectors. The vectors of the table 1570 can result in a second (rich) set 1502 of ECG values.

Locations of ECG sensing electrodes such as those in the table 1590 can be implemented by the support structure in a number of ways. It will be appreciated that such may eliminate the need for adhesive gelled electrodes placed individually based on anatomical references. Rather, the support structure may be adjustable in a proportional way, in the horizontal and the vertical direction, which may therefore maintain the relative position of electrodes with respect to each other, regardless of the actual distance among them that the body will dictate. Examples are now described.

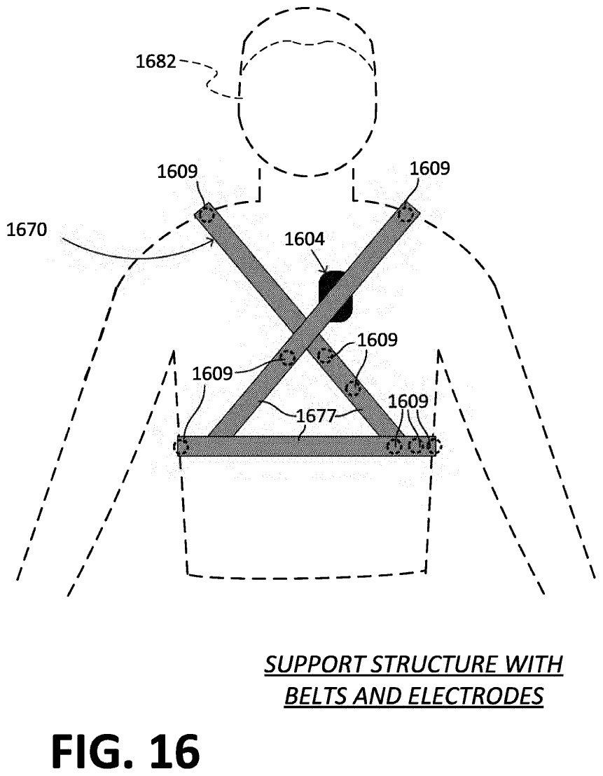

FIG. 16 is a diagram of a sample support structure 1670 for a WMS that is implemented using a system of belts 1677 according to embodiments. The belts 1677 are worn by a person 1682 as shown. The person 1682 has a chest that is shown, and a back that is not shown. The belts 1677 support ECG sensing electrodes 1609, and are arranged so as to contact the person 1682 when the person is wearing the belts 1677. Additional ECG sensing electrodes may be supported by the belts 1677 in the back of the person 1682. Defibrillation electrodes are optionally also supported by the belts 1677. In the example of FIG. 16, a defibrillation electrode 1604 is shown at the chest of the person 1682, while another (not shown) might be at their back, as seen in the embodiments of FIG. 4. A challenge with the belt approach can be that extra care should be taken so that the belts 1677 exert enough tension on the upper electrodes to get good connection. Worse, the belts 1677 themselves may generate noise. In addition, larger ECG sensing electrodes may result in less noise in the ECG signal, which is why a vest system may be preferred, especially for smaller patients.

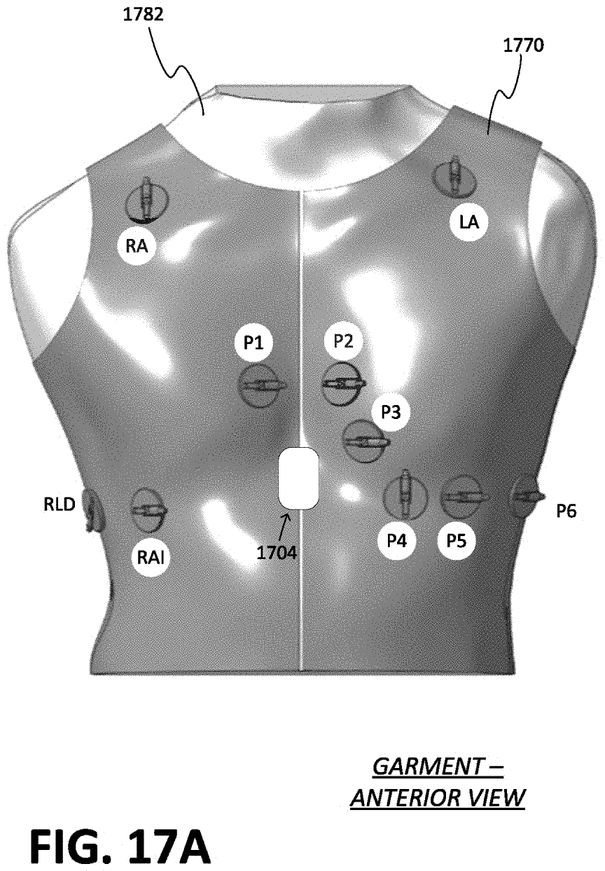

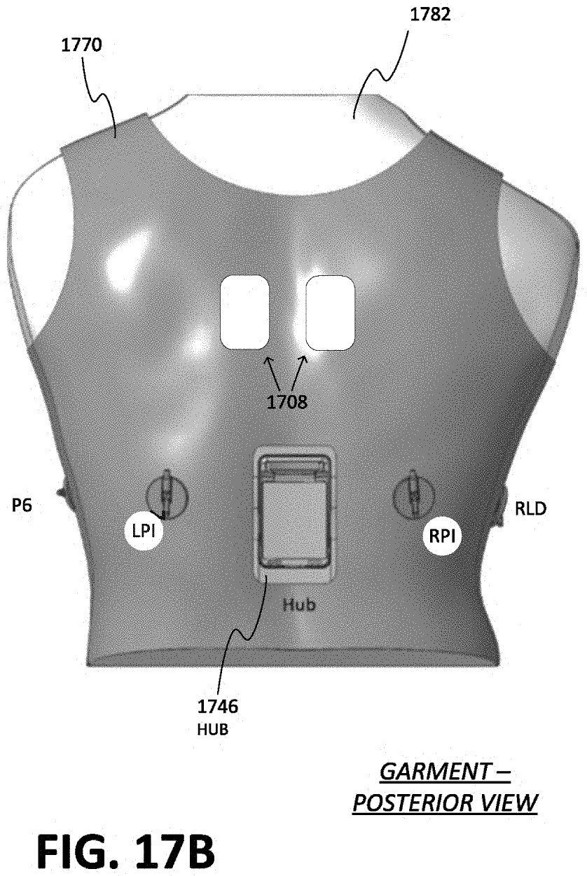

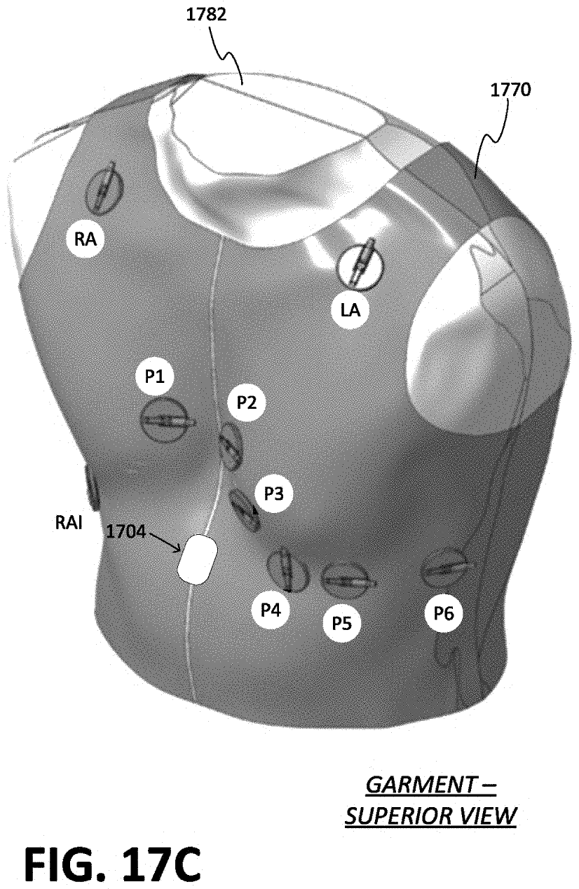

FIG. 17A is an anterior (front) view of a sample support structure for a WMS that is implemented by a vest 1770, according to embodiments. The vest 1770 is configured to be worn by a person, and is shown with reference to portions of a torso 1782. The vest 1770 can be made of breathable fabric.

The vest 1770 has defibrillation electrodes 1704, 1708. The vest 1770 also has ECG sensing electrodes such as those listed in the table 1590. These ECG sensing electrodes can be placed at the locations shown in FIG. 17A and in FIG. 17B of this document. These ECG sensing electrodes are configured to contact the patient when the patient is wearing the vest 1770. This can be implemented by the ECG sensing electrodes protruding enough in the inside of the vest to make the contact with the patient. In some embodiments, the electrodes are cushioned. An advantage is that, by the patient putting on the vest 1770, the leads are placed on them automatically. Defibrillation electrodes may optionally also be supported by the vest 1770. For long term monitoring applications, the wearer can be provided with two garments, so that the other garment can be washed while one is being worn.

FIG. 17B is a posterior (rear) view of the support structure that is implemented by the vest 1770 of FIG. 17A. As can be seen, the defibrillation electrode 1708 in implemented in two parts. FIG. 17B also shows a hub 1746 that can be electrically connected to the ECG sensing electrodes. The hub 1746 then could gather the ECG and other data for download. The vest 1770 optionally also has a pocket (not shown) for receiving therein the hub 1746. The electrical connection of the hub 1746 to the ECG electrodes can be wireless, or wired for instance with conductive wires that are attached to the vest 1770, embedded between fabric layers of the garment, and so on. The hub 1746 may also include a 3-axis accelerometer, attached directly or in a pocket of the vest 1770. For inpatient monitoring, the accelerometer could be used to notify nursing staff of patient motion and position to help prevent falls, and step counting for rehabilitation. In WCD uses, the hub 1746 can be connected to a unit (not shown), for instance as described in FIG. 4.

FIG. 17C is a superior (perspective) view of the support structure that is implemented by the vest 1770 of FIG. 17A.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

Methods are now described, which have operations some of which may be implemented by one or more devices that include logic circuitry.

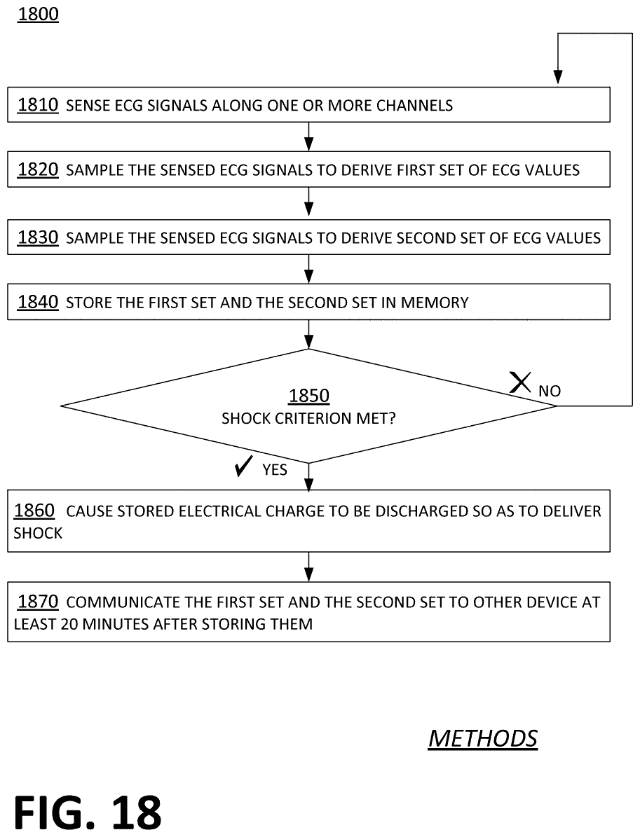

FIG. 18 shows a flowchart 1800 for describing methods according to embodiments. According to an operation 1810, ECG signals of the ambulatory patient may be sensed. The sensing can be performed by the ECG sensing electrodes, along one or more channels.

According to another operation 1820, the sensed ECG signals are sampled to produce a first set of ECG values. The first set may have a first number of ECG values per unit time.

According to another operation 1830, the sensed ECG signals are sampled to produce a second set of ECG values. The second set may have a first number of ECG values per unit time. The second number can be at least twice as large as the first number, or even larger.

In some embodiments, the operation 1830 may start being performed when switched on. For instance, as already mentioned, it may be further detected whether or not a starting condition is met, similarly to what was described with reference to the decision diamond 1152, and so on.

In some embodiments, the operation 1830 may stop being performed when switched off. For instance, as already mentioned, it may be further detected whether or not a stopping condition is met, similarly to what was described with reference to the decision diamond 1159, and so on.

According to another operation 1840, the first set of ECG values and the second set of ECG values are stored in a memory.

According to another operation 1850, it may be determined whether or not a shock criterion is met. The determination may be made by a processor, from one of the first set of ECG values and the second set of ECG values. If the answer is NO, then execution may return to another operation, such as the operation 1810.

If at the operation 1860 the answer is YES then, at least some of the stored electrical charge can be caused by the processor to be discharged via the therapy electrode through the ambulatory patient. The discharge can be while the support structure is worn by the ambulatory patient, so as to deliver a shock to the ambulatory patient.

According to another operation 1870, the first set of ECG values and the second set of ECG values can be communicated to an other device that is distinct from the unit that contains the processor. The communicating can be performed by the communication module at least 20 minutes after the operation 1840.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

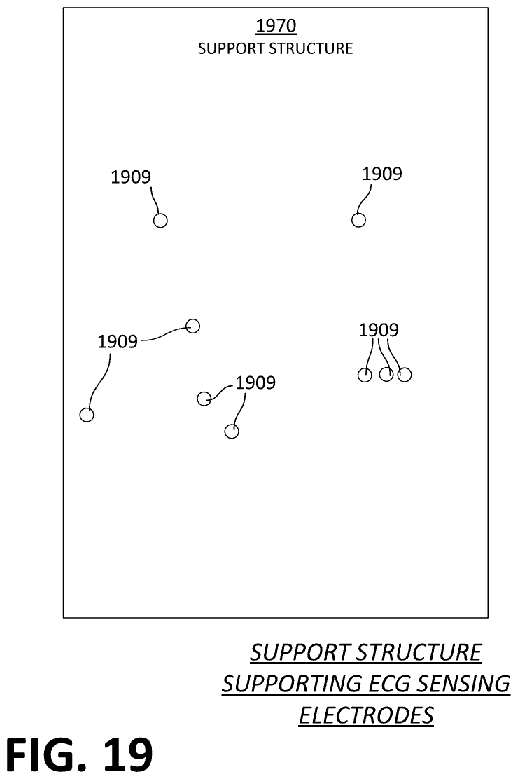

Referring now to FIG. 19, embodiments further include a WMS that supports only the ECG sensing devices, but is not configured to implement a WCD. These include the support structure as a stand-alone wearable, which is for a person who has not necessarily been characterized as a patient for a WCD. For instance, the support structure can be for a person for multiple ECG monitoring functions including in-patient monitoring, advanced (5-minute resting ECG), Holter monitoring, treadmill tests, sports applications, etc. In the example of FIG. 19, a support structure 1970 supports multiple ECG sensing electrodes 1909, for instance seven or more. Similarly with FIG. 14, these ECG sensing electrodes 1909 may be placed at any desirable locations, and FIG. 19 does not speak as to the positions of these electrodes. The support structure 1970 can be made by using belts in addition to other components, a vest, and so on. More particular examples are now described, which are drawn from the above.

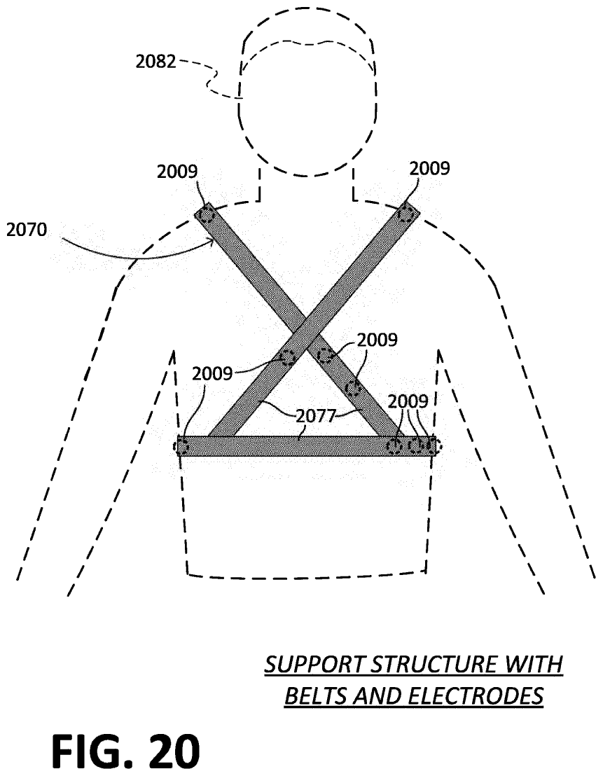

FIG. 20 is a diagram of a sample support structure 2070 for a WMS that is implemented using a system of belts 2077 according to embodiments. The belts 2077 are configured to be worn by a person 2082, for instance as shown. The person 2082 has a chest that is shown, and a back that is not shown. The belts 2077 support ECG sensing electrodes 2009, and are arranged so as to contact the person 2082 when the person is wearing the belts 2077. Additional ECG sensing electrodes may be supported by the belts 2077 in the back of the person 2082.

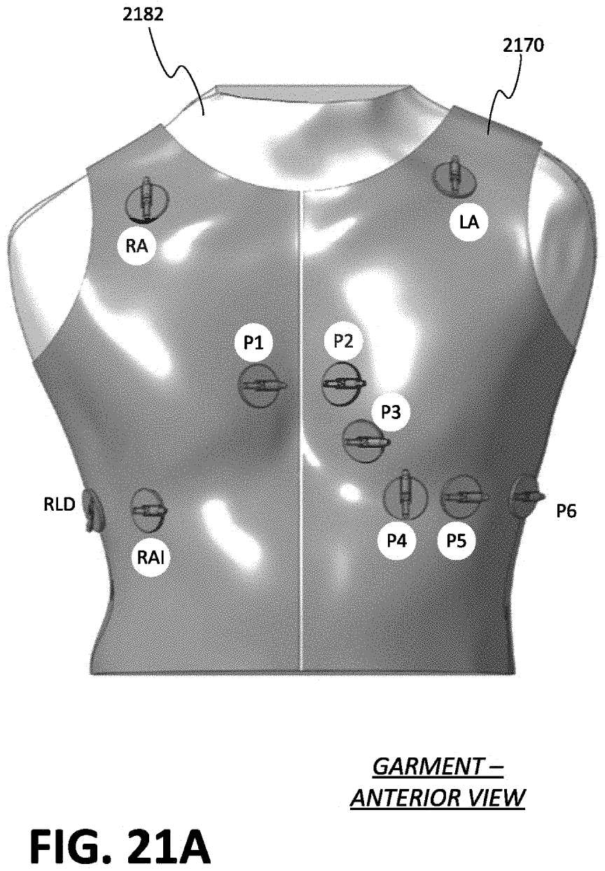

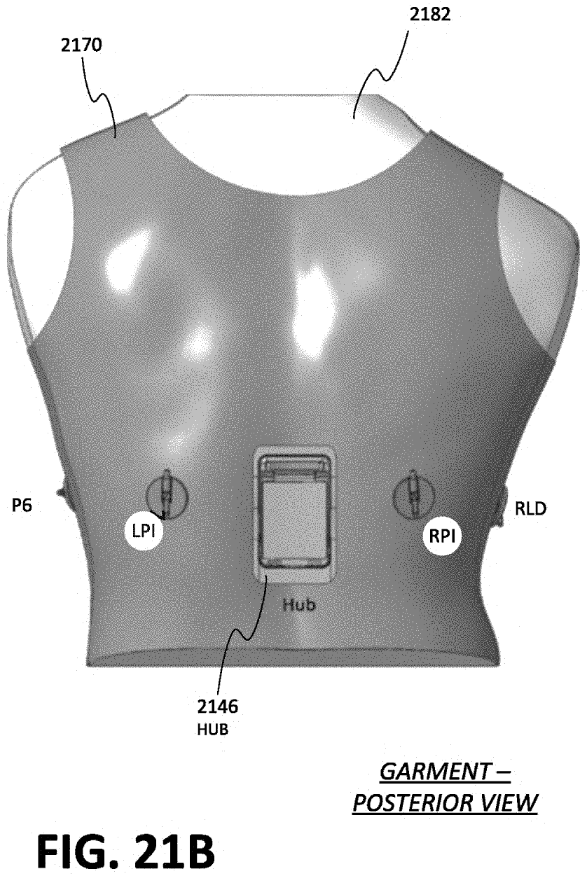

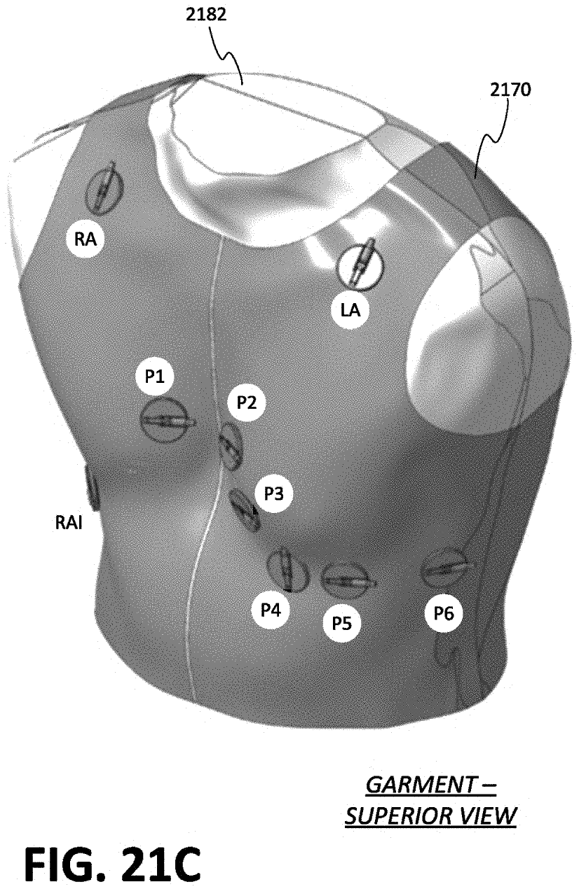

FIG. 21A is an anterior (front) view of a sample support structure for a WMS that is implemented by a vest 2170, according to embodiments. The vest 2170 is configured to be worn by a person, as shown with reference to portions of a torso 2182. The vest 2170 can be made of as described for the vest 1770.

These ECG sensing electrodes can be placed at the locations shown in FIG. 21A and in FIG. 21B of this document. The vest 2170 has ECG sensing electrodes such as those listed in the table 1590. These ECG sensing electrodes are configured to contact the patient when the patient is wearing the vest 2170.

FIG. 21B is a posterior (rear) view of the support structure that is implemented by the vest 2170 of FIG. 21A. FIG. 21B also shows a hub 2146 that can be electrically connected to the ECG sensing electrodes, and can be as described for the hub 1746. The vest 2170 optionally also has a pocket (not shown) for receiving therein the hub 2146. The electrical connection of the hub 2146 to the ECG electrodes can be wireless, or wired for instance with conductive wires that are attached to the vest 2170, embedded between fabric layers of the garment, and so on. The hub 2146 may also include a 3-axis accelerometer, attached directly or in a pocket of the vest 2170. For inpatient monitoring, the accelerometer could be used to notify nursing staff of patient motion and position to help prevent falls, and step counting for rehabilitation.

FIG. 21C is a superior (perspective) view of the support structure that is implemented by the vest 2170 of FIG. 21A.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, element, component or process that are identical, or at least similar or related. Where made, such a further effort was not required, but was nevertheless made gratuitously so as to facilitate comprehension by the reader. Even where made in this document, such a further effort might not have been made completely consistently for all of the versions or embodiments that are made possible by this description. Accordingly, the description controls in defining an item, aspect, element, component or process, rather than its reference numeral. Any similarity in reference numerals may be used to infer a similarity in the text, but not to confuse aspects where the text or other context indicates otherwise.

The claims of this document define certain combinations and subcombinations of elements, features and acts or operations, which are regarded as novel and non-obvious. The claims also include elements, features and acts or operations that are equivalent to what is explicitly mentioned. Additional claims for other such combinations and subcombinations may be presented in this or a related document. These claims are intended to encompass within their scope all changes and modifications that are within the true spirit and scope of the subject matter described herein. The terms used herein, including in the claims, are generally intended as “open” terms. For example, the term “including” should be interpreted as “including but not limited to,” the term “having” should be interpreted as “having at least,” etc. If a specific number is ascribed to a claim recitation, this number is a minimum but not a maximum unless stated otherwise. For example, where a claim recites “a” component or “an” item, it means that the claim can have one or more of this component or this item.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1. A wearable medical system (“WMS”) for a patient, including at least:
a support structure configured to be worn by the patient;
ECG (Electrocardiogram) sensing electrodes configured to sense ECG signals of the patient along one or more channels;
a unit configured to be maintained on a body of the patient when the support structure is worn by the patient;
an energy storage module configured to store an electrical charge;
a therapy electrode coupled to the energy storage module and configured to be maintained on the body of the patient when the support structure is worn by the patient;
a memory in the unit;
a processor in the unit, the processor configured to:
store in the memory:
a) a first set of ECG values produced by sampling the sensed ECG signals, the first set having a first number of ECG values per unit time, and
b) a second set of ECG values produced by sampling the sensed ECG signals, the second set having a second number of ECG values per unit time, the second number at least twice as large as the first number,
determine from at least one of the first set of ECG values and the second set of ECG values whether or not a shock criterion is met, and
cause, responsive to the shock criterion being met, at least some of the stored electrical charge to be discharged via the therapy electrode through the patient while the support structure is worn by the patient so as to deliver a shock to the patient; and
a communication module configured to communicate the first set of ECG values and the second set of ECG values to another device, the other device distinct from the unit.

2. The WMS of claim 1, in which:
the second number is at least five times larger than the first number.

3. The WMS of claim 1, in which:
at a certain time moment, the processor is configured to store the first set of ECG values,
and to concurrently store the second set of ECG values.

4. The WMS of claim 3, in which:
portions of the first set of ECG values are stored in first sectors of the memory,
portions of the second set of ECG values are stored in second sectors of the memory, and
the second sectors are not interspersed among the first sectors.

5. The WMS of claim 1, in which:
at a certain time moment, the processor is configured to store either the first set of ECG values or the second set of ECG values but not both.

6. The WMS of claim 5, in which:
portions of the first set of ECG values are stored in first sectors of the memory,
portions of the second set of ECG values are stored in second sectors of the memory, and
at least some of the second sectors are interspersed among the first sectors.

7. The WMS of claim 1, in which the processor is further configured to:
store in the memory additional ECG values of the second set, responsive to causing the at least some of the stored electrical charge to be thus discharged, within 10 sec from thus causing.

8. The WMS of claim 1, in which the processor is further configured to:
detect whether a starting condition is met, and
in response to the starting condition being met, start storing the second set of ECG values.

9. The WMS of claim 8, in which:
the processor is further configured to detect, while storing the first set, noise in the one or more channels that is above a noise threshold, and
in response to detecting noise above the noise threshold, indicate that the starting condition is met.

10. The WMS of claim 8, in which:
the processor is further configured to detect an arrhythmia from the first set of ECG values, and
in response to detecting the arrhythmia, indicate that the starting condition is met.

11. The WMS of claim 8, further including:
an input device configured to be actuated by the patient, and
in which: the starting condition is met responsive to the input device being actuated by the patient.

12. The WMS of claim 8, further including:
a clock configured to render a time input, and
in which: the starting condition is met responsive to the time input meeting a suitability criterion.

13. The WMS of claim 1, in which the processor is further configured to:
detect whether a stopping condition is met, and
in response to the stopping condition being met, stop storing the second set of ECG values.

14. The WMS of claim 13, further including:
a motion detector configured to render a motion detection input, and
in which: the stopping condition is met responsive to the motion detection input meeting an unrest criterion.

15. The WMS of claim 13, further including:
a battery that is configured to be inserted into the unit to power the processor, the battery configured to store an electrical charge and to supply the stored electrical charge to the energy storage module, and
in which: the processor is further configured to determine a charge level of the electrical charge stored in the battery, and
the stopping condition is met responsive to the inputted charge level being below a threshold.

16. The WMS of claim 1, in which:
the first set of ECG values is produced by sampling the sensed ECG signals at a first sampling rate,
the second set of ECG values is produced by sampling the sensed ECG signals at a second sampling rate, and
the second sampling rate is at least 50% faster than the first sampling rate.

17. The WMS of claim 16, in which:
the second sampling rate is at least 740 ECG values per sec.

18. The WMS of claim 1, in which:
the first set of ECG values is produced by sampling the ECG signals that are sensed along at least one but no more than six of the one or more channels, but
the second set of ECG values is produced by sampling the ECG signals that are sensed concurrently along at least seven of the one or more channels.

19. The WMS of claim 18, in which:
the second set of ECG values are produced by sampling the ECG signals that are sensed concurrently along 12 of the one or more channels.

20. The WMS of claim 18, in which:
the first set of ECG values is produced by sampling the sensed ECG signals at a first sampling rate,
the second set of ECG values is produced by sampling the sensed ECG signals at a second sampling rate, and
the second sampling rate is at least 50% faster than the first sampling rate.

21. The WMS of claim 1, in which:
the second set of ECG values are produced by sampling the ECG signals that are sensed concurrently along at least 16 of the one or more channels.

22. (canceled)

23. The WMS of claim 1, in which:
the support structure has at least 12 ECG sensing electrodes.

24-52. (canceled)
